(19) United States
(12) Patent Application Publication
(10) Pub. No.: US 2025/0257332 A1
(43) Pub. Date: Aug. 14, 2025

(54) TRUNCATED AND FUSION PROTEINS

(71) Applicant: Monash University, Clayton (AU)

(72) Inventors: Peter CURRIE, Clayton, Victoria (AU); Mikael MARTINO, Clayton, Victoria (AU)

(21) Appl. No.: 18/704,817

(22) PCT Filed: Oct. 27, 2022
(86) PCT No.: PCT/AU2022/051290

§ 371 Date: Apr. 25, 2024

(30) Foreign Application Priority Data
Oct. 27, 2021 (AU) 2021903439

(51) Int. Cl. C12N 9/10; A61K 38/00; A61P 19/00; A61P 37/06; C07K 14/475

(52) U.S. Cl. CPC C12N 9/1077; A61P 19/00; A61P 37/06; C07K 14/475; A61K 38/00; C07K 2319/33; C07K 2319/74; C12Y 204/02012

(57) ABSTRACT

The present invention relates to productive tissue repair and regeneration. and in particular poylpeptides. compositions including said polypeptides. and methods of using said polypeptides or compositions for productive tissue repair and regeneration. In one aspect. the invention provides a polypeptide comprising. consisting essentially of or consisting of a C-terminal portion of NAMPT comprising a truncated cytokine finger motif (cif) motif. In another aspect. the present invention provides a fusion protein comprising. consisting essentially of or consisting of a polypeptide of a full length NAMPTcif or truncated variants and a tissue delivery or retention enhancing moiety.

### FIELD OF THE INVENTION

The present invention relates to productive tissue repair and regeneration, and in particular polypeptides, compositions including said polypeptides, and methods of using said polypeptides or compositions for productive tissue repair and regeneration.

### RELATED APPLICATION

This application claims priority from Australian provisional application no. 2021903439 filed 27 Oct. 2021, the entire contents of which are herein incorporated by reference in its entirety.

### BACKGROUND OF THE INVENTION

Skeletal muscle typically forms approximately 40% of a body mass in human adults. It is formed during development by myogenesis wherein paired blocks of paraxial mesoderm known as somites give rise to a transitory myotome that forms muscle stem cells and expands to form an integrated and complex musculature through fusion of myoblasts to the surface of myotubes. In a further stage of myogenesis, muscle stem cells (called satellite cells) migrate to occupy a niche between the sarcolemma and basal lamina of individual myofibers. Amniotes are born with a full set of muscle fibres and, in adults, muscle repair is generally effected through an increase in the size of existing fibres. Throughout life, homeostasis, growth, regeneration and repair of muscle tissue is driven by mesoderm derived skeletal muscle resident stem cells. At a molecular level, quiescent satellite cells require and express the transcription factor PAX7 and also express PAX3. Following skeletal muscle damage, some satellite cells become activated, proliferate to form myoblasts that differentiate and fuse to form new myofibres or merge with and repair damaged muscle fibres. This myogenic programme is governed by myogenic regulatory factors, MYF5, MYOD, MYOG and MRF4. A wealth of other factors and cells associated with the muscle niche are thought to be involved in the complex cellular processes and final production of functional tissue in homeostatic and regenerative contexts. Hence it has been difficult to date to identify the source and nature of the signals that stimulate satellite cell activation and proliferation.

The satellite cell is archetypal of a unipotent tissue-resident stem cell that occupies a specific anatomical niche within a differentiated tissue. Decades of research have revealed the extraordinary capacity of this system to effectively coordinate muscle repair in response to a wide variety of insults. Despite this demonstrated regenerative capacity, transplantation of isolated muscle stem cells has yet to provide therapeutic impact, and pro-regenerative treatments that stimulate muscle stem cells are entirely lacking at this juncture.

There is a need for new and/or improved compositions and methods for use in myoblast based therapy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a truncated or modified NAMPT cytokine finger (cif) polypeptide.

In this aspect, the present invention provides a NAMPT polypeptide fragment comprising, consisting essentially of or consisting of a C-terminal portion of NAMPT comprising a truncated cif motif. Preferably, the only amino acid sequence of the polypeptide that is derived from or has homology or identity to the NAMPT protein is the truncated cif motif.

In this aspect, the present invention provides a polypeptide comprising, consisting essentially of or consisting of a C-terminal portion of NAMPT comprising a truncated cif motif. Preferably, the only amino acid sequence of the polypeptide that is derived from or has homology or identity to the NAMPT protein is the truncated cif motif.

In any embodiment, the polypeptide binds to CCR5 and/or stimulates muscle progenitor proliferation.

In any embodiment, the only amino acid sequence of the polypeptide that binds to CCR5 and/or stimulates muscle progenitor stimulation is the truncated cif motif.

In any embodiment, the cif motif comprises or consists of the amino acid sequence set out in any one of SEQ ID Nos: 1, 2 or 3. Preferably, the NAMPT polypeptide comprises, consists essentially of or consists of a truncated amino acid sequence of SEQ ID NO: 1.

In any embodiment, the truncation of the cif motif may be an N-terminal truncation and/or a C-terminal truncation. In one embodiment, the N- and/or C-terminal truncation is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids. Preferably, the truncation is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids N- and/or C-terminal of the amino acid sequence of, or equivalent to, SEQ ID NO: 1.

In any embodiment, the truncation is of the N-terminal residues 1-12, 1-20, 1-28 or 1-35 of the cif motif. Preferably, the truncation is of, or equivalent to, the N-terminal residues 1-12, 1-20, 1-28 or 1-35 of the amino acid sequence set forth in SEQ ID NO: 1.

In one embodiment, the polypeptide comprises, consists essentially of or consists of an amino acid sequence of any one of SEQ ID Nos: 4, 5, 6, 7, 8, 9, 10 or 11, or an amino acid sequence that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 4, 5, 6, 7, 8, 9, 10 or 11. Alternatively, the polypeptide comprises, consists essentially of or consists of an amino acid sequence of any one of SEQ ID Nos: 4, 5, 6, 7, 8, 9, 10 or 11, or an amino acid sequence that is equal to, or at least, 70%, 75%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID Nos: 4, 5, 6, 7, 8, 9, 10 or 11.

In one embodiment, % identity or identical to a sequence means that the polypeptide has the same length, for example number of amino acids, but the amino acids across that length are only 70%, 75%, 80%, 85%, 86%, 87%, 88%, 89%, 90%, 91%, 92%, 93%, 94%, 95%, 96%, 97%, 98%, 99%, or 100% identical. Typically, the only differences in amino acid identity is a result of conservative substitutions (for example those outlined in Table 3 below).

In one embodiment, the polypeptide is equal to, or less than, about 110, about 109, about 108, about 107, about 106, about 105, about 104, about 103, about 102, about 101, about 100, about 99, about 98, about 97, about 96, about 95, about 94, about 93, about 92, about 91, about 90, about 89, about 88, about 87, about 86, about 85, about 84, about 83, about 82, about 81, about 80, about 79, about 78, about 77, about 76, about 75, about 74, about 73, about 72, about 71, about 70, about 69, about 68, about 67, about 66, about 65, about 64, about 63, about 62, about 61, about 60, about 59, about 58, about 57, or about 56 amino acids in length.

In one embodiment, the polypeptide is equal to, or less than, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, or 56 amino acids in length.

In one embodiment, the amino acid sequence of the polypeptide that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 4, 5, 6, 7, 8, 9, 10 or 11 is equal to, or less than, about 110, about 109, about 108, about 107, about 106, about 105, about 104, about 103, about 102, about 101, about 100, about 99, about 98, about 97, about 96, about 95, about 94, about 93, about 92, about 91, about 90, about 89, about 88, about 87, about 86, about 85, about 84, about 83, about 82, about 81, about 80, about 79, about 78, about 77, about 76, about 75, about 74, about 73, about 72, about 71, about 70, about 69, about 68, about 67, about 66, about 65, about 64, about 63, about 62, about 61, about 60, about 59, about 58, about 57, or about 56 amino acids in length.

In one embodiment, the amino acid sequence of the polypeptide that is equal to, or at least, 70%, 75%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID Nos: 4, 5, 6, 7, 8, 9, 10 or 11 is equal to, or less than, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, or 56 amino acids in length.

In any embodiment, the polypeptide or fusion protein comprises an amino acid sequence having 1, 2, 3, 4, 5, 6, 7 or 8 conservative (for example those outlined in Table 3 below) or non-conservative amino acid substitutions, deletions or additions to the above sequences, and retains CCR5 or tissue stem cell interacting activity. Preferably the conservative or non-conservative amino acid substitutions, deletions or additions are not of the amino acids 431 to 435 or 472 to 491 (numbering corresponding to human NAMPT, e.g. SEQ ID NO: 19).

In one embodiment, the polypeptide comprises, consists essentially of or consists of the amino acid sequence of SEQ ID NO: 8 with an N-terminal truncation. Preferably the N-terminal truncation is less than 6 amino acids.

In one embodiment, any polypeptide as described herein comprises the C-terminal alpha helix present in the cif motif.

In one embodiment, the polypeptide consists of an amino acid sequence having equal to, or at least, 85% sequence identity to the amino acid sequence of SEQ ID NO: 4 or 5. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 4 or 5.

In one embodiment, the polypeptide consists of an amino acid sequence having equal to, or at least, 85% sequence identity to the amino acid sequence of SEQ ID NO: 6 or 7. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 6 or 7.

In one embodiment, the polypeptide consists of an amino acid sequence having equal to, or at least, 85% sequence identity to the amino acid sequence of SEQ ID NO: 8 or 9. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 8 or 9.

In one embodiment, the polypeptide consists of an amino acid sequence having equal to, or at least, 85% sequence identity to the amino acid sequence of SEQ ID NO: 10 or 11. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to SEQ ID NO: 10 or 11.

In one embodiment, the truncated NAMPT cytokine finger (cif) polypeptide has an N terminal truncation of, or equivalent to, residues 402 to 413 of SEQ ID NO: 1, and wherein the polypeptide has at least 85% sequence identity to the amino acid sequence of 414 to 491 of SEQ ID NO: 1. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 414 to 491 of SEQ ID NO: 1.

In one embodiment, the truncated NAMPT cytokine finger (cif) polypeptide has an N terminal truncation of, or equivalent to, residues 402 to 421 of SEQ ID NO: 1, and wherein the polypeptide has at least 85% sequence identity to the amino acid sequence of 422 to 491 of SEQ ID NO: 1. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 422 to 491 of SEQ ID NO: 1.

In one embodiment, the truncated NAMPT cytokine finger (cif) polypeptide has an N terminal truncation of, or equivalent to, residues 402 to 429 of SEQ ID NO: 1, and wherein the polypeptide has at least 85% sequence identity to the amino acid sequence of 430 to 491 of SEQ ID NO: 1. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 430 to 491 of SEQ ID NO: 1.

In one embodiment, the truncated NAMPT cytokine finger (cif) polypeptide has an N terminal truncation of, or equivalent to, residues 402 to 435 of SEQ ID NO: 1, and wherein the polypeptide has at least 85% sequence identity to the amino acid sequence of 436 to 491 of SEQ ID NO: 1. Preferably, the amino acid sequence of the polypeptide is equal to, or at least, 86%, 87%, 88%, 89%, 90%, 91%, 92%, 93%, 94%, 95%, 96%, 97%, 98%, 99%, or 100% identical to the amino acid sequence of 436 to 491 of SEQ ID NO: 1.

In one embodiment, the polypeptide may be in monomeric, dimeric or multimeric form. To facilitate dimerization any polypeptide described herein may be modified to allow homo or heterodimerisation. The modification may be the addition of an amino acid, either natural or non-natural, that forms a covalent bond, for example as cysteine that forms a disulphide bond. Typically, the addition of an amino acid, such as a cysteine, that forms a covalent bond is at the N- or C-terminus of the polypeptide. In one embodiment, the polypeptide comprises, consists essentially of or consists of an amino acid sequence of any one of SEQ ID Nos: 4, 5, 6, 7, 8, 9, 10 or 11 with an additional cysteine at the N- or C-terminus.

In another embodiment, there is provided a dimeric polypeptide formed by covalent bonded, preferably disulphide bonded, monomers of a polypeptide described herein.

In another embodiment, the polypeptide comprises, consists essentially of or consists of 2 or more truncated cif motif as described herein. Preferably, the 2 or more truncated cif motifs are separated by a linker.

The peptide linker may be any one or more repeats of Gly-Gly-Ser (GGS), Gly-Gly-Gly-Ser (GGGS) or Gly-Gly-Gly-Gly-Ser (GGGGS) or variations thereof. In one embodiment, the linker may comprise or consist of the sequence GGGGSGGGGSGGGGS (G4S) 3. In one embodiment, the peptide linker can include the amino acid sequence GGGGS (a linker of 6 amino acids in length) or even longer. The linker may a series of repeating glycine and serine residues (GS) of different lengths, i.e., (GS) n where n is any number from 1 to 15 or more. For example, the linker may be (GS) 3 (i.e., GSGSGS) or longer (GS) 11 or longer. It will be appreciated that n can be any number including 1, 2, 3, 4, 5, 6, 7, 8, 9, 10, 11 or more.

In another aspect, the present invention provides a fusion protein comprising, consisting essentially of or consisting of a polypeptide of the invention as described herein and a tissue delivery or retention enhancing moiety such as one or more ECM and/or other tissue specific binding moieties.

In another aspect, the present invention provides a fusion protein comprising, consisting essentially of or consisting of a full length NAMPT cytokine finger (cif) polypeptide and a tissue delivery or retention enhancing moieties such as one or more ECM and/or other tissue specific binding moieties. In one embodiment, the full length NAMPT cytokine finger (cif) polypeptide comprises, consists essentially of or consists of an amino acid sequence as set forth in SEQ ID NO: 1.

In any embodiment, the ECM binding moiety binds to any one or more of the following ECM molecules: collagen, fibronectin, tenascin C, osteopontin, fibrinogen, and heparan sulfate proteoglycans.

In any embodiment, the ECM binding moiety is derived from placenta growth factor (PIGF), amphiregulin (Areg), collagenase (col) or von Willebrand factor (vWF). Preferably, the PLGF, Areg, col or vWF is human.

In any embodiment, the ECM binding moiety comprises, consists essentially of or consists of positively charged amino acid residues. Preferably, a contiguous sequence of positively charged amino acid residues. In one embodiment, the positively charged residues comprises, consists essentially of or consists of RRRPK, RKKK, KRRR or any others described herein including in SEQ ID NO: 12 and 13. In another embodiment, the ECM binding moiety comprises at least 2 contiguous sequences of positively charged amino acid residues.

In any embodiment, the ECM binding moiety comprises, consists essentially of or consist of any one of SEQ ID Nos: 12 to 16. In another embodiment, the ECM binding moiety comprises, consists essentially of or consist of an amino acid sequence that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 12 to 16 wherein the ECM binding moiety binds to one or more ECM proteins with the same affinity, an affinity not significantly different, or an affinity of at least 80%, 95%, 90%, 91%, 92%, 93%, 94%, 95%, 96%, 97%, 98% or 99%, an ECM binding moiety of anyone or SEQ ID Nos: 12 to 16 from which it was derived.

In one embodiment an extracellular matrix (ECM) binding moiety known in the art is included. Illustrative ECM binding peptides are described in US publication no. 2014/0011978 and US publication no. 20140010832. Standard methods are used to conjugate agents or peptides to moieties such as ECM binding moieties with or without linkers.

In one embodiment, polypeptide or fusion proteins described herein comprise one or more signalling enhancing moieties such as a syndecan binding moiety. Typically, a syndecan binding moiety is included to provide tonic or enhanced CCR5 signalling via syndecans.

In any embodiment, a polypeptide or fusion protein as described herein binds to satellite cells and stimulates satellite cell activation, myoblast proliferation and/or muscle regeneration.

In one embodiment, the fusion protein comprises, consists essentially of or consists of the amino acid sequence of any one of SEQ ID NO: 12 to 16 fused, linked, or linked directly to the amino acid sequence of any one of SEQ ID Nos: 1 to 11. Preferably, the fusion protein comprises, consists essentially of or consists of the amino acid sequence of any one of SEQ ID NO: 12 to 16 fused, linked, or linked directly to the amino acid sequence of SEQ ID Nos: 1. Preferably, the fusion protein comprises, consists essentially of or consists of, in N to C terminal arrangement, the amino acid sequence of any one of SEQ ID NO: 12 to 16 fused, linked, or linked directly to the amino acid sequence of any one of SEQ ID Nos: 1 to 11. In one embodiment, the protein comprises, consists essentially of or consists of the amino acid sequence encodes by any one of SEQ ID Nos: 29 to 31.

In any embodiment, a polypeptide or fusion protein as described herein stimulates muscle progenitor cell (e.g. myoblast) proliferation to a level that is equal to or greater than full length NAMPT, for example comprising, consisting essentially of or consisting of SEQ ID NO: 19, or equal to or greater than full length NAMPTcif, for example comprising, consisting essentially of or consisting of SEQ ID NO: 1 or 2. Preferably, muscle progenitor cell (e.g. myoblast) proliferation is determined by an assay described herein, including in Example 1.

In any embodiment, a polypeptide or fusion protein described herein stimulates TLR4 activation to a significantly lower level than full length NAMPT. For example, stimulates TLR4 activation to a level equal to, or less than, 90%, 85%, 80%, 75%, 70%, 65%, 60%, 55%, 50%, 45%, 40%, 35%, 30%, 25%, 20%, 15% or 10% full length NAMPT (for example, a polypeptide comprising or consisting of an amino acid sequence of SEQ ID NO: 19). In any embodiment, a polypeptide or fusion protein stimulates TLR4 activation to the same, or not-significantly different, level as the cytokine finger motif of NAMPT (for example, a polypeptide consisting of the amino acid sequence of SEQ ID NO: 1). The level of TLR4 activation may be determined using an assay described herein, including in Example 1.

In another aspect, the present invention also provides isolated nucleic acids encoding a polypeptide or fusion protein as described herein, for example, comprising, consisting essentially of or consisting of the amino acid sequence of any one of SEQ ID Nos: 4, 5, 6, 7, 8, 9, 10, 11, 17 or 18. In one embodiment, a nucleic acid molecule encoding a polypeptide or fusion protein described herein comprises, consists essentially of or consists of the polynucleotide sequence set out in any one of SEQ ID NO: 25 to 31, or a polynucleotide sequence that has at least 70%, 75%, 80%, 85%, 86%, 87%, 88%, 89%, 90%, 91%, 92%, 93%, 94%, 95%, 96%, 97%, 98%, 99%, or 100% sequence identity thereto.

The nucleic acid molecule may be an RNA or DNA or RNA:DNA or a chemically modified form thereof. For example, the nucleic acid may be in the form of a viral or non-viral vector.

In another aspect, the present invention provides vectors comprising said nucleic acids, optionally, operably linked to control sequences.

In another aspect, the present invention provides host cells containing the vectors, and methods for producing and optionally recovering the polypeptides or fusion proteins.

In another aspect, the present invention provides a cell expressing a polypeptide or fusion protein as described herein.

In another aspect, the present invention provides compositions providing chemokine receptor interaction or binding activity or muscle tissue stem cell interacting activity for use in stimulating muscle regeneration. In one embodiment, the present application provides compositions providing chemokine receptor interaction or binding activity or satellite cell binding or interacting activity for use in stimulating muscle regeneration without fibrosis or substantially without fibrosis. In one embodiment, the chemokine receptor is a CCR5 chemokine receptor or a tissue stem cell receptor that binds NAMPT, including a tissue stem cell receptor that binds NAMPTcif. In one embodiment the composition, comprising a cell or other agent that provides CCR5 interacting activity, binds to tissue stem cells, particularly muscle stem cells.

In one embodiment, the composition comprises a polypeptide, fusion protein, nucleic acid, vector, or cell as described herein and a pharmaceutically acceptable carrier, diluent or excipient. In one embodiment, the composition may further comprise one, two or all of (a) a tissue stem cell (such as a satellite cell) or precursor therefore or progeny thereof, (b) a macrophage or a precursor therefore or progeny thereof, and (c) a scaffold or retentive material.

In one non-limiting embodiment, promoting muscle stem cell chemokine receptor signalling is particularly useful in treating subjects with a muscle injury including volumetric muscle loss injuries or muscle degeneration/atrophy, or muscular or neuromuscular impairments, muscular or neuromuscular degenerative conditions, myopathy, or the propensity therefore. In one embodiment, chemokine receptor binding activity is provided in the form of a cell such as a macrophage or stem cell expressing a polypeptide or fusion protein as described herein.

In another aspect, the present invention provides a method of stimulating proliferation of a stem cell, such as satellite cell proliferation, the method comprising administering to a cell or subject an effective amount of a polypeptide, fusion protein, composition or cell as described herein, thereby stimulating proliferation of a stem cell.

In one embodiment, the chemokine receptor is a CCR5 receptor.

In one embodiment, the CCR5 receptor is a tissue stem cell or tissue stem cell progeny CCR5 receptor. In one embodiment the CCR5 receptor is a satellite cell or satellite cell progeny CCR5 receptor.

In one embodiment, in vitro, in vivo and ex vivo applications are contemplated.

In another aspect, the present invention provides a method of stimulating muscle tissue regeneration in a subject, the method comprising administering to a muscle of the subject an effective amount of a polypeptide, fusion protein, composition or cell as described herein, thereby stimulating muscle tissue regeneration.

In any embodiment, the polypeptide or fusion protein is a CCR5 agonist. That is, it stimulates receptor signalling or downstream events such as satellite cell activation and proliferation. In one embodiment, the polypeptide or fusion protein specifically activates tissue stem cells. In one embodiment, the polypeptide or fusion protein specifically activates satellite cells.

As described herein, in one embodiment, tissue regeneration stimulated by the method is associated with minimal fibrosis. Thus, in another aspect, the present application provides polypeptides, fusion proteins, compositions, cells and methods for reducing fibrosis development in a patient or biological tissue subject to regenerative treatment.

In one embodiment, the present application provides a method suitable for regenerating muscle tissue in vitro, in vivo or ex vivo. Accordingly, a polypeptide, fusion protein, composition or cell described herein is proposed for use in stem cell based therapies and tissue engineering. In another embodiment a polypeptide, fusion protein, composition or cell described herein is for use in artificial meat production in vitro.

In another aspect, the present invention provides a method of stimulating muscle tissue regeneration, the method comprising administering to a muscle an effective amount of a polypeptide, fusion protein, composition or cell as described herein, wherein the a polypeptide, fusion protein, composition or cell as described herein binds to satellite cells and stimulates satellite cell activation, myoblast proliferation and muscle regeneration and the absence of substantial fibrosis (scar formation).

In another aspect, the present invention provides a method of stimulating muscle tissue regeneration in a subject where inflammation is undesirable, the method comprising administering to a muscle an effective amount of a polypeptide, fusion protein, composition or cell as described herein, wherein the polypeptide, fusion protein, composition or cell as described herein, thereby stimulating muscle tissue regeneration in this subject.

In one embodiment, the inflammation that is undesirable is inflammation mediated by TLR activation, preferably TLR4 activation.

In one embodiment, the subject may be diagnosed with an inflammatory myopathy. Exemplary inflammatory myopathies include polymyositis, dermatomyositis, inclusion body myositis, necrotizing autoimmune myopathy.

In this aspect, the present invention provides a method of treating an inflammatory myopathy in a subject, the method comprising administering to a muscle of the subject an effective amount of a polypeptide, fusion protein, composition or cell as described herein, thereby treating an inflammatory myopathy. Preferably the inflammatory myopathy is polymyositis, dermatomyositis, inclusion body myositis or necrotizing autoimmune myopathy.

Reference to NAMPT and CCR5 includes homologues and orthologs thereof include from any animal including mammals, non-mammalian vertebrates, fish and birds.

In one embodiment, the present application provides method of stimulating muscle tissue regeneration, the method comprising administering to a muscle an effective amount of a composition comprising a cell comprising or encoding a polypeptide or fusion protein as described herein, and optionally a component that enhances delivery to or retention in the muscle, wherein the polypeptide or fusion protein as described herein binds to satellite cells and stimulates myoblast proliferation and muscle regeneration.

In one embodiment, the cell is a macrophage. In one embodiment the macrophage is isolated from tissue. In one embodiment, the macrophage is induced from stem cells such as bone marrow precursors or iPSC. In one embodiment, the macrophage or macrophage precursor (a monocyte) is isolated from a supply tissue such as, but not limited to blood, lymph, bone marrow) and then subjected to in vitro cell or tissue culture to induce the desired tissue niche directed phenotype. In one embodiment, the cell composition is cryopreserved and/or contains a delivery agent.

As known in the art, macrophages may be generated in vitro from stem cells by various means. Macrophages generated from stem cells, such as BMSC, in the presence of IFNγ or LPS are generally considered as “inflammatory” macrophages referred to as “M1 macrophages.” Those generated in the presence of IL-4 or IL-10 have what is called a “pro-resolution” activity and are referred to as “M2” macrophages.

In one embodiment, the subject macrophage expresses M2 macrophage markers.

In one embodiment, the macrophage cell expresses one or two or three or four or five of mmp9, arg2, mmp 13a, L-plastin and cd163.

In one other embodiment, the macrophage subset expresses prox1a and pou2f3.

In one embodiment, the composition further comprises a stem cell and/or a macrophage cell.

In one embodiment, the stem cell is a satellite cell. In another embodiment the stem cell is a unipotent or multipotent stem cell.

In one embodiment of the method, the active ingredient or main ingredient is, or is only, a polypeptide or fusion protein described herein, or a pharmaceutically acceptable salt, hydrate, homolog, ortholog, tautomer, stereoisomer, pro-drug thereof.

Pro-drugs refer to agents that can be converted via some chemical or physiological process (e.g., enzymatic processes and metabolic hydrolysis) to a polypeptide or fusion protein described herein. Thus, the term “prodrug” also refers to a precursor of a biologically active compound that is pharmaceutically acceptable. A prodrug may be inactive when administered to a subject but is converted in vivo to an active compound. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively.

In one embodiment, the polypeptide or fusion protein described herein further comprises one or more moieties such as a linker, stability enhancing, signalling enhancing, delivery enhancing or label moiety.

In another aspect, the present invention provides compositions comprising a polypeptide, fusion protein, nucleic acid, vector, or cell as defined herein. Pharmaceutical and physiologically active compositions are provided. Cellular compositions are expressly provided.

In one embodiment, the cell is a macrophage. In one embodiment the macrophage is isolated from tissue. In one embodiment, the macrophage is induced from stem cells such as bone marrow precursors or iPSC. In one embodiment, the macrophage or macrophage precursor (a monocyte) is isolated from a supply tissue such as, but not limited to blood, lymph, bone marrow) and then subjected to in vitro cell or tissue culture to induce the desired tissue niche directed phenotype. In one embodiment, the cell composition is cryopreserved and/or contains a delivery agent.

As known in the art, macrophages may be generated in vitro from stem cells by various means. Macrophages generated from stem cells, such as BMSC, in the presence of IFNg or LPS are generally considered as “inflammatory” macrophages referred to as “M1 macrophages.” Those generated in the presence of IL-4 or IL-10 have what is called a “pro-resolution” activity and are referred to as “M2” macrophages.

In one embodiment, the subject macrophage expresses M2 macrophage markers.

In one embodiment, the macrophage cell expresses one or two or three or four or five of mmp9, arg2, mmp13a, L-plastin and cd163.

In one other embodiment, the macrophage subset expresses prox1a and pou2f3.

In one embodiment, the composition comprises or is administered together with a supporting material such as a hydrogel, glue, foam or retentive material, scaffold etc. Delicate structures are generally suitable for enabling more delicate tissue regeneration. As examples, materials can be used which are quite rapidly absorbed, such as certain fibrin, collagen, hydrogel and alginate formulations. Alternatively, slowly absorbable synthetics can be used, such as poly-4-hydroxybutarate. Silk fibers or even substantially smooth products derived from mammalian origin such as muscle extracellular matrix are also contemplated. Non-absorbable synthetics, such as polypropylene and polyethylene, provide support and reliability. In one embodiment the composition comprises a fibrin hydrogel. In another embodiment, RAFT-acrylamide based support surfaces are provided to enhance tissue regeneration and bioavailability of a polypeptide, fusion protein, nucleic acid, vector, or cell to the target site.

In one embodiment there is provided a composition comprising a polypeptide, fusion protein, nucleic acid, vector, or cell as described herein and any one or two or three or four of: (i) a satellite cell or precursor therefore or progeny thereof (ii) a macrophage or a precursor therefore or progeny thereof (iii) a scaffold (semi-solid or solid support) or retentive material (iv) a tissue delivery enhancing or cell retention moiety.

In one embodiment the scaffold or retentive material is a hydrogel, such as a fibrin or acrylamide hydrogel. In one embodiment, the tissue delivery enhancing or cell retention moiety is an ECM-binding moiety.

As used herein, except where the context requires otherwise, the term “comprise” and variations of the term, such as “comprising”, “comprises” and “comprised”, are not intended to exclude further additives, components, integers or steps.

By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements (e.g., 1, 2, 3, 4, 5, 6, 7, 8, 9, 10 additional amino acid residues at the N-terminus or C-terminus of a polypeptide sequence) are optional and may or may not be present depending upon whether or not they affect the activity or action of the listed elements.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Mouse volumetric muscle loss injury NAMPT supplementation. A-D, Local delivery of NAMPT promotes muscle regeneration in an adult mouse muscle injury model (schematic, I). (B) Volumetric muscle defects were created and directly treated with NAMPT delivered via a fibrin hydrogel. Masson's trichrome stained representative tissue sections of murine rectus femoris (RF) muscle (10 days post treatment) through the middle of the defect, demonstrate NAMPT delivery significantly increased the regenerated muscle area (dark red, quantification, C) while simultaneously showing a significant reduction in fibrotic tissue (purple/blue, white dashed line demarcates the separation between fibrotic and healthy muscle fibres, while the fascia surrounding the muscle is stained in blue (quantification, D)). C-D, Means±SEM. One-way ANOVA with Dunnett's post hoc test for multiple comparisons (n=5 mice per group). E-I, Satellite cells demonstrated enhanced proliferation upon exogenous NAMPT supplementation. Mouse muscle injuries were treated with NAMPT (0.5 μg) delivered in fibrin or fibrin only control. (E-F) The total number of satellite cells (PAX7⁺) (E) and the number of satellite cells in the proliferation phase (PAX7⁺/Ki67⁺) (F) was quantified by flow cytometry in tissues harvested 4 days post treatment. The graphs show the fractions of satellite cells per 10,000 cells in the harvested tissue (n=6 mice for the fibrin group, n=5 mice for NAMPT-treated group). (G) Representative muscle regenerate cryosections stained for PAX7 (satellite cells, yellow), wheat germ agglutinin (WGA, magenta), and nuclei (DAPI, blue) for tissues harvested 6 days post treatment. (H-I) Centrally nucleated muscle fibres were quantified at 6 days post treatment (n=6 mice per group) (H). Representative histology tissue sections with Haematoxylin and Eosin (I). E-F, H, Mean±S.E.M. Two-tailed Student's t-test.

FIG. 2. NAMPT binds to the CCR5 receptor present on muscle stem cells and induces proliferation. (A) Exogenous NAMPT supplementation enhances myoblast proliferation. In vitro assay assessing the effects of exogenously introduced factors on C2C12 myoblast proliferation. Proliferation is identified by EdU incorporation. NAMPT administration (2 commercially available NAMPT sources tested, hrNAMPT₍₁₎ and hrNAMPT₍₂₎) leads to a dose dependent increase in myoblast proliferation. This effect is specifically mediated via the CCR5 receptor. Co-administration of NAMPT with the CCR2/CCR5 dual inhibitor cenicriviroc (CVC) and CCR5 specific inhibitor maraviroc (MVC) abolishes NAMPT's pro-proliferative response, while co-administration with the CCR2 inhibitor PF-4136309 (PF) does not hinder NAMPT's stimulatory effect on myoblast proliferation. In agreement with this finding, CCR5′s endogenous ligands mrCCL8 and mrCCL4 functioned to enhance C2C12 proliferation while the CCR2-specific ligand mrCCL2 failed to increase proliferative rates beyond that of the control. NAMPT's pro-proliferative function is separate from its intracellular role in energy metabolism, as co-administering NAMPT with a NAMPT enzymatic inhibitor GMX1778 does not impact its effect on myoblast proliferation. Mean+S.D. Two-way ANOVA with Tukey's multiple comparison test. (B-C) The C-terminal fragment of NAMPT regulates cytokine activity. (B) NAMPT contains a “cytokine finger” (cif) conserved in other cytokines. (C) NAMPTcif inhibits the binding of NAMPT to CCR5. Mean±SEM

FIG. 3. Human NAMPT cytokine finger N-terminally truncated variants. Predicted structure of human NAMPT cytokine finger (hNAMPTcif) variants designed by N-terminal truncation of regions containing positively charged amino acids. (A) Human NAMPTcif (residues 402-491 of full length NAMPT, e.g. SEQ ID NO: 19); (B) hNAMPTcif-T1 (residues 414-491 of full length NAMPT) removes the N-terminus beta strand, loop and subsequent beta strand (C) hNAMPTcif-T2 (residues 422-491 of full length NAMPT) starting from T1 removes the N-terminus loop and short helix; including one lysine (D) hNAMPTcif-T3 (residues 430-491 of full length NAMPT) starting from T2 removes the N-terminus loop; including 3 lysines, and two arginines (E) hNAMPTcif-T4 (residues 436-491 of full length NAMPT) starting from T3 removes the N-terminus beta strand and loop; including a histidine. (F) Human NAMPT cytokine finger and its truncated variants stimulate muscle progenitor proliferation. C2C12 mouse myoblasts were treated with 10 nM of full length NAMPT (FL-NAMPT), human NAMPT cytokine finger (hNAMPTcif) or N-terminally truncated variants of hNAMPTcif (hNAMPTcif-T1, hNAMPTcif-T2, hNAMPTcif-T3, where each increase in number represents a shorter hNAMPTcif fragment) for 48 hours. Quantification of cell proliferation was performed using CyQuant Proliferation Assay kit. Data are displayed as percentage increase vs. PBS-treated negative control. 10% foetal bovine serum (FBS) was used as positive control. n=3-6 technical replicates per conditions, 2 independent experiments. Asterisks denote significance for one-sample t-tests where *: p<0.05 and **: p<0.01. # denotes p<0.05 and ## denotes p<0.01 for Mann-Whitney tests versus FL-hNAMPT.

FIG. 4. ECM-binding domain-fused NAMPTcif retains pro-proliferative activity. C2C12 mouse myoblasts were treated with 2 nM, 10 nM or 20 nM NAMPTcif (NAMPTcif) or NAMPTcif fused with an N-terminal heparin-binding sequence derived from placental growth factor 2 (PIGF-NAMPTcif) for 48 hours. Quantification of cell proliferation was performed using CyQuant Proliferation Assay kit. Data are displayed as percentage increase vs. PBS-treated negative control. Mean±SEM. n=4 independent experiments. Two-tailed unpaired t-test. n.s.: not statistically significant.

FIG. 5. NAMPTcif does not induce TLR4 activity compared to full length NAMPT. HEK-Blue TLR4 reporter cells were treated with 19 nM NAMPTcif or NAMPTcif fused with an N-terminal heparin-binding sequence derived from placental growth factor 2 (PIGF-NAMPTcif) for 24 hours. 5 ug/ml polymyxin B was added to each treatment well to abrogate signalling induced by any traces of endotoxin. Quantification of TLR4 activation was determined against a standard curve generated by bacterial lipopolysaccharide (LPS) serial dilutions and reported as equivalent LPS concentration. Mean±SEM. n=2 independent experiments with triplicate wells per condition. One-way ANOVA with Dunnett's multiple comparisons test. *: p<0.05.

FIG. 6. Human NAMPT “cytokine finger” N-terminally truncated variants. Predicted structure of human NAMPT “cytokine finger” variants designed by N- and C-terminal truncation of regions containing structural elements and/or positively charged amino acids. Structures are denoted by amino acid number corresponding to full length human NAMPT, e.g SEQ ID NO: 19.

FIG. 7. Human NAMPT variants stimulate muscle progenitor proliferation. C2C12 mouse myoblasts were treated with 20 nM full length NAMPT (NAMPT) or NAMPT variants for (NAMPTₓₓₓ₋ₓₓₓ, where xxx are amino acid numbers) 48 hours. Quantification of cell proliferation was performed using CyQuant Proliferation Assay kit. Data are displayed as percentage increase vs. PBS-treated negative control. Box and whiskers denote median with minimum and maximum values. n=4-5 independent experiments per protein. Statistical significance is indicated as exact p-value. Significant increases in proliferation per condition were analysed by one-sample t-test. Significance between conditions were analysed by one-way ANOVA with Tukey post-hoc test.

FIG. 8. Human NAMPT variants stimulate human satellite cell proliferation. Human primary satellite cells were treated with 20 nM full length NAMPT (NAMPT) or NAMPT variants (NAMPTₓₓₓ₋ₓₓₓ, where xxx are amino acid numbers) for 48 hours. The media contained the growth factors IGF-1 and FGF-2 as well. Quantification of cell proliferation was performed using CyQuant Proliferation Assay kit. Data are displayed as percentage increase vs. PBS-treated negative control. Treatment with 20% FBS was used as a positive control. Box and whiskers denote median with minimum and maximum values. n=6-10 independent experiments per protein. Statistical significance is indicated as p-value and determined by one-sample t-test.

FIG. 9. Human NAMPT variants stimulate human endothelial cell proliferation. Human primary endothelial cells derived from umbilical vein were treated with 20 nM full length NAMPT (NAMPT) or NAMPT variants (NAMPTₓₓₓ₋ₓₓₓ, where xxx are amino acid numbers) for 48 hours. Quantification of cell proliferation was performed using CyQuant Proliferation Assay kit. Data are displayed as percentage increase vs. PBS-treated negative control. Treatment with 10% FBS was used as a positive control. Box and whiskers denote median with minimum and maximum values. n=6 independent experiments per protein. Statistical significance is indicated as p-value and determined by one-sample t-test.

FIG. 10. Minimal versions of NAMPT protein enhance proliferation in response to muscle injury in zebrafish larvae. Treatment with NAMPT₄₀₂₋₄₉₁ and NAMPT₄₂₂₋₄₉₁ following needle-stick muscle injury induces a significant increase in cell proliferation within the injury zone. The smallest version of NAMPT (NAMPT₄₂₂₋₄₉₁), stimulates cell proliferation specifically in the wound at significantly higher levels as compared to human recombinant-NAMPT (hrNAMPT). Representative images are shown in panel (A). Violin plots in (B) show number of EdU-positive cells in the ‘injury’ zone and ‘external’ represent the two-adjacent somites encompassing the injury zone, (n=22 control, n=9 hrNAMPT-treated, n=6 NAMPT₄₀₂₋₄₉₁ and n=8 NAMPT₄₂₂₋₄₉₁). The thick black lines and dashed black lines within the violin plot indicate the median and quartiles, respectively. Two-way ANOVA with Tuckey's multiple comparison test.

### SUMMARY OF SEQUENCE LISTING

TABLE 1
Sequence of the invention.

| SEQ ID NO: | DESCRIPTION | SEQUENCE |
|---|---|---|
| SEQ ID NO: 1 | Illustrative Human | SYVVTNGLGINVFKDPVADPNKRSKKGRLSLHRTPA |
| | cytokine finger | GNFVTLEEGKGDLEEYGQDLLHTVFKNGKVTKSYSF |
| | (NAMPT402-491, | DEIRKNAQLNIELEAAHH |
| | numbering corresponds | |
| | to full length NAMPT, | |
| | e.g SEQ ID NO: 19) | |
| SEQ ID NO: 2 | Human NAMPT cif | CSYVVTNGLGINVFKDPVADPNKRSKKGRLSLHRTP |
| | monomer as base for | AGNFVTLEEGKGDLEEYGQDLLHTVFKNGKVTKSYS |
| | dimeric or multimeric | FDEIRKNAQLNIELEAAHH |
| | form, with, in this | |
| | embodiment, an n- | |
| | terminal cysteine to | |
| | facilitate dimer | |
| | formation | |
| SEQ ID NO: 3 | Mouse C-terminal | SYVVTNGLGVNVFKDPVADPNKRSKKGRLSLHRTPA |
| | fragment of NAMPT as | GNFVTLEEGKGDLEEYGHDLLHTVFKNGKVTKSYSF |
| | CCR5 interacting | DEVRKNAQLNIEQDVAPH |
| | peptide | |
| SEQ ID NO: 4 | Truncated hNAMPTcif- | FKDPVADPNKRSKKGRLSLHRTPAGNFVTLEEGKGD |
| | T1 (NAMPT414-491, | LEEYGQDLLHTVFKNGKVTKSYSFDEIRKNAQLNIE |
| | numbering corresponds | LEAAHH |
| | to full length NAMPT, | |
| | e.g SEQ ID NO: 19) | |
| SEQ ID NO: 5 | Truncated hNAMPTcif- | HHHHHHHHENLYFQGFKDPVADPNKRSKKGRLSLHR |
| | T1 (NAMPT 414-491, | TPAGNFVTLEEGKGDLEEYGQDLLHTVFKNGKVTKS |
| | numbering corresponds | YSFDEIRKNAQLNIELEAAHH |
| | to full length NAMPT, | |
| | e.g SEQ ID NO: 19) | |
| | (including HIS tag) | |
| SEQ ID NO: 6 | Truncated hNAMPTcif- | NKRSKKGRLSLHRTPAGNFVTLEEGKGDLEEYGQDL |
| | T2 (NAMPT 422-491, | LHTVFKNGKVTKSYSFDEIRKNAQLNIELEAAHH |
| | numbering corresponds | |
| | to full length NAMPT, | |
| | e.g SEQ ID NO: 19) | |
| SEQ ID NO: 7 | Truncated hNAMPTcif- | HHHHHHHHENLYFQGNKRSKKGRLSLHRTPAGNFVT |
| | T2 (NAMPT 422-491, | LEEGKGDLEEYGQDLLHTVFKNGKVTKSYSFDEIRK |
| | numbering corresponds | NAQLNIELEAAHH |
| | to full length NAMPT, | |
| | e.g SEQ ID NO: 19) | |
| | (including HIS tag) | |
| SEQ ID NO: 8 | Truncated hNAMPTcif- | LSLHRTPAGNFVTLEEGKGDLEEYGQDLLHTVFKNG |
| | T3 (NAMPT430-491, | KVTKSYSFDEIRKNAQLNIELEAAHH |
| | numbering corresponds | |
| | to full length NAMPT, | |
| | e.g SEQ ID NO: 19) | |
| SEQ ID NO: 9 | Truncated hNAMPTcif- | HHHHHHHHENLYFQGLSLHRTPAGNFVTLEEGKGDL |
| | T3 (NAMPT430-491, | EEYGQDLLHTVFKNGKVTKSYSFDEIRKNAQLNIEL |
| | numbering corresponds | EAAHH |
| | to full length NAMPT, | |
| | e.g SEQ ID NO: 19) | |
| | (including HIS tag) | |
| SEQ ID NO: 10 | Truncated hNAMPTcif- | PAGNFVTLEEGKGDLEEYGQDLLHTVFKNGKVTKSY |
| | T4 (NAMPT 436-491, | SFDEIRKNAQLNIELEAAHH |
| | numbering corresponds | |
| | to full length NAMPT, | |
| | e.g SEQ ID NO: 19) | |
| SEQ ID NO: 11 | Truncated hNAMPTcif- | HHHHHHHHENLYFQGPAGNFVTLEEGKGDLEEYGQD |
| | T4 (NAMPT 436-491, | LLHTVFKNGKVTKSYSFDEIRKNAQLNIELEAAHH |
| | numbering corresponds | |
| | to full length NAMPT, | |
| | e.g SEQ ID NO: 19) | |
| | (including HIS tag) | |
| SEQ ID NO: 12 | Human PIGF123-141 | RRRPKGRGKRRREKQRPTD |
| | (ECM-binding domain | |
| | derived from placenta | |
| | growth factor) | |
| SEQ ID NO: 13 | Human Areg25-42 | RKKKGGKNGKNRR |
| | (ECM-binding domain | |
| | derived from | |
| | amphiregulin) | |
| SEQ ID NO: 14 | Human von Willebrand | WREPSFMALS |
| | factor (VWF) ECM | |
| | binding moiety 1 | |
| SEQ ID NO: 15 | Human von Willebrand | CSQPLDVILLLDGSSSFPASYFDEMKSFAKAFISKA |
| | factor (VWF) ECM | NIGPRLTQVSVLQYGSITTIDVPWNVVPEKAHLLSL |
| | binding moiety 2 | VDVMQREGGPSQIGDALGFAVRYLTSEMHGARPGAS |
| | | KAVVILVTDVSVDSVDAAADAARSNRVTVFPIGIGD |
| | | RYDAAQLRILAGPAGDSNVVKLQRIEDLPTMVTLGN |
| | | SFLHKLCSGFVRICTG |
| SEQ ID NO: 16 | Human Collagenase | TKKTLRT |
| | ECM binding moiety | |
| SEQ ID NO: 17 | Human PIGF123- | RRRPKGRGKRRREKQRPTDCSYVVTNGLGVNVFKDP |
| | 141/mouse NAMPTcif | VADPNKRSKKGRLSLHRTPAGNFVTLEEGKGDLEEY |
| | with Cys | GHDLLHTVFKNGKVTKSYSFDEVRKNAQLNIEQDVA |
| | | PH |
| SEQ ID NO: 18 | Human Areg25- | RKKKGGKNGKNRRCSYVVTNGLGVNVFKDPVADPNK |
| | 42/mouse NAMPTcif | RSKKGRLSLHRTPAGNFVTLEEGKGDLEEYGHDLLH |
| | with Cys | TVFKNGKVTKSYSFDEVRKNAQLNIEQDVAPH |
| SEQ ID NO: 19 | Example of Human | MNPAAEAEFNILLATDSYKVTHYKQYPPNTSKVYSY |
| | NAMPT as CCR5 | FECREKKTENSKLRKVKYEETVFYGLQYILNKYLKG |
| | interacting | KVVTKEKIQEAKDVYKEHFQDDVFNEKGWNYILEKY |
| | polypeptide | DGHLPIEIKAVPEGFVIPRGNVLFTVENTDPECYWL |
| | | TNWIETILVQSWYPITVATNSREQKKILAKYLLETS |
| | | GNLDGLEYKLHDFGYRGVSSQETAGIGASAHLVNFK |
| | | GTDTVAGLALIKKYYGTKDPVPGYSVPAAEHSTITA |
| | | WGKDHEKDAFEHIVTQFSSVPVSVVSDSYDIYNACE |
| | | KIWGEDLRHLIVSRSTQAPLIIRPDSGNPLDTVLKV |
| | | LEILGKKFPVTENSKGYKLLPPYLRVIQGDGVDINT |
| | | LQEIVEGMKQKMWSIENIAFGSGGGLLQKLTRDLLN |
| | | CSFKCSYVVTNGLGINVFKDPVADPNKRSKKGRLSL |
| | | HRTPAGNFVTLEEGKGDLEEYGQDLLHTVFKNGKVT |
| | | KSYSFDEIRKNAQLNIELEAAHH |
| SEQ ID NO: 20 | Example of Mouse | MNAAAEAEFNILLATDSYKVTHYKQYPPNTSKVYSY |
| | NAMPT as CCR5 | FECREKKTENSKVRKVKYEETVFYGLQYILNKYLKG |
| | interacting | KVVTKEKIQEAKEVYREHFQDDVFNERGWNYILEKY |
| | polypeptide | DGHLPIEVKAVPEGSVIPRGNVLFTVENTDPECYWL |
| | | TNWIETILVQSWYPITVATNSREQKKILAKYLLETS |
| | | GNLDGLEYKLHDFGYRGVSSQETAGIGASAHLVNFK |
| | | GTDTVAGIALIKKYYGTKDPVPGYSVPAAEHSTITA |
| | | WGKDHEKDAFEHIVTQFSSVPVSVVSDSYDIYNACE |
| | | KIWGEDLRHLIVSRSTEAPLIIRPDSGNPLDTVLKV |
| | | LDILGKKFPVTENSKGYKLLPPYLRVIQGDGVDINT |
| | | LQEIVEGMKQKKWSIENVSFGSGGALLQKLTRDLLN |
| | | CSFKCSYVVTNGLGVNVFKDPVADPNKRSKKGRLSL |
| | | HRTPAGNFVTLEEGKGDLEEYGHDLLHTVFKNGKVT |
| | | KSYSFDEVRKNAQLNIEQDVAPH |
| SEQ ID NO: 21 | Example of a Human | agctatgttgtaactaatggccttgggattaacgtc |
| | cDNA NAMPT cif | ttcaaggacccagttgctgatcccaacaaaaggtcc |
| | fragment encoding | aaaaagggccgattatctttacataggacgccagca |
| | sequence | gggaattttgttacactggaggaaggaaaaggagac |
| | | cttgaggaatatggtcaggatcttctccatactgtc |
| | | ttcaagaatggcaaggtgacaaaaagctattcattt |
| | | gatgaaataagaaaaaatgcacagctgaatattgaa |
| | | ctggaagcagcacatcatta |
| SEQ ID NO: 22 | Mouse cDNA encoding | agctatgttgtaaccaatggccttggggttaatgtg |
| | NAMPTcif fragment | tttaaggacccagttgctgatcccaacaaaaggtca |
| | | aaaaagggccggttatctttacataggacaccagcg |
| | | gggaactttgttacacttgaagaaggaaaaggagac |
| | | cttgaggaatatggccatgatcttctccatacggtt |
| | | ttcaagaatgggaaggtgacaaaaagctactcattt |
| | | gatgaagtcagaaaaaatgcacagctgaacatcgag |
| | | caggacgtggcacctcatt |
| SEQ ID NO: 23 | Example of Human DNA | atgaatcctgcggcagaagccgagttcaacatcctc |
| | encoding NAMPT full | ctggccaccgactcctacaaggttactcactataaa |
| | length | caatatccacccaacacaagcaaagtttattcctac |
| | | tttgaatgccgtgaaaagaagacagaaaactccaaa |
| | | ttaaggaaggtgaaatatgaggaaacagtattttat |
| | | gggttgcagtacattcttaataagtacttaaaaggt |
| | | aaagtagtaaccaaagagaaaatccaggaagccaaa |
| | | gatgtctacaaagaacatttccaagatgatgtcttt |
| | | aatgaaaagggatggaactacattcttgagaagtat |
| | | gatgggcatcttccaatagaaataaaagctgttcct |
| | | gagggctttgtcattcccagaggaaatgttctcttc |
| | | acggtggaaaacacagatccagagtgttactggctt |
| | | acaaattggattgagactattcttgttcagtcctgg |
| | | tatccaatcacagtggccacaaattctagagagcag |
| | | aagaaaatattggccaaatatttgttagaaacttct |
| | | ggtaacttagatggtctggaatacaagttacatgat |
| | | tttggctacagaggagtctcttcccaagagactgct |
| | | ggcataggagcatctgctcacttggttaacttcaaa |
| | | ggaacagatacagtagcaggacttgctctaattaaa |
| | | aaatattatggaacgaaagatcctgttccaggctat |
| | | tctgttccagcagcagaacacagtaccataacagct |
| | | tgggggaaagaccatgaaaaagatgcttttgaacat |
| | | attgtaacacagttttcatcagtgcctgtatctgtg |
| | | gtcagcgatagctatgacatttataatgcgtgtgag |
| | | aaaatatggggtgaagatctaagacatttaatagta |
| | | tcgagaagtacacaggcaccactaataatcagacct |
| | | gattctggaaaccctcttgacactgtgttaaaggtt |
| | | ttggagattttaggtaagaagtttcctgttactgag |
| | | aactcaaagggttacaagttgctgccaccttatctt |
| | | agagttattcaaggggatggagtagatattaatacc |
| | | ttacaagagattgtagaaggcatgaaacaaaaaatg |
| | | tggagtattgaaaatattgccttcggttctggtgga |
| | | ggtttgctacagaagttgacaagagatctcttgaat |
| | | tgttccttcaagtgtagctatgttgtaactaatggc |
| | | cttgggattaacgtcttcaaggacccagttgctgat |
| | | cccaacaaaaggtccaaaaagggccgattatcttta |
| | | cataggacgccagcagggaattttgttacactggag |
| | | gaaggaaaaggagaccttgaggaatatggtcaggat |
| | | cttctccatactgtcttcaagaatggcaaggtgaca |
| | | aaaagctattcatttgatgaaataagaaaaaatgca |
| | | cagctgaatattgaactggaagcagcacatcatta |
| SEQ ID NO: 24 | Mouse cDNA encoding | atgaatgctgcggcagaagccgagttcaacatcctg |
| | NAMPT full-length | ctggccaccgactcgtacaaggttactcactataaa |
| | | caatacccacccaacacaagcaaagtttattcctac |
| | | tttgaatgccgtgaaaagaagacagaaaactccaaa |
| | | gtaaggaaggtgaaatacgaggaaacagtattttat |
| | | gggttgcagtacattcttaataagtacttaaaaggt |
| | | aaagtagtgaccaaagagaaaatccaggaggccaaa |
| | | gaagtgtacagagaacatttccaagatgatgtcttt |
| | | aacgaaagaggatggaactacatccttgagaaatac |
| | | gatggtcatctcccgattgaagtaaaggctgttccc |
| | | gagggctctgtcatccccagagggaacgtgctgttc |
| | | acagtggaaaacacagacccagagtgctactggctt |
| | | accaattggattgagactattcttgttcagtcctgg |
| | | tatccaattacagtggccacaaattccagagaacag |
| | | aagagaatactggccaaatatttgttagaaacctct |
| | | ggtaacttagatggtctggaatacaagttacatgac |
| | | tctggttacagaggagtctcttcgcaagagactgct |
| | | ggcataggggcatctgctcatttggttaacttaaaa |
| | | ggaacagatactgtggcgggaattgctctaattaaa |
| | | aaatactatgggacaaaagatcctgttccaggctat |
| | | tctgttccagcagcagagcacagtaccataacggct |
| | | tgggggaaagaccatgagaaagatgcttttgaacac |
| | | atagtaacacagttctcatcagtgcctgtgtctgtg |
| | | gtcagcgatagctatgacatttataatgcgtgtgag |
| | | aaaatatggggtgaagacctgagacatctgatagta |
| | | tcgagaagtacagaggcaccactaatcatcagacct |
| | | gactctggaaatcctcttgacactgtattgaaggtc |
| | | ttagatattttaggcaagaagtttcctgttactgag |
| | | aactcaaaaggctacaagttgctgccaccttatctt |
| | | agagtcattcaaggagatggcgtggatatcaatact |
| | | ttacaagagattgtagagggaatgaaacaaaagaag |
| | | tggagtatcgagaatgtctccttcggttctggtggc |
| | | gctttgctacagaagttaacccgagacctcttgaat |
| | | tgctccttcaagtgcagctatgttgtaaccaatggc |
| | | cttggggttaatgtgtttaaggacccagttgctgat |
| | | cccaacaaaaggtcaaaaaagggccggttatcttta |
| | | cataggacaccagcggggaactttgttacacttgaa |
| | | gaaggaaaaggagaccttgaggaatatggccatgat |
| | | cttctccatacggttttcaagaatgggaaggtgaca |
| | | aaaagctactcatttgatgaagtcagaaaaaatgca |
| | | cagctgaacatcgagcaggacgtggcacctcatt |
| SEQ ID NO: 25 | Example of nucleotide | TTCAAAGACCCGGTTGCTGACCCGAACAAACGTTCT |
| | sequence of | AAAAAAGGTCGTCTGTCTCTGCACCGTACCCCGGCT |
| | hNAMPTcif-T1 | GGTAACTTCGTTACCCTGGAAGAAGGTAAAGGTGAC |
| | | CTGGAAGAATACGGTCAGGACCTGCTGCACACCGTT |
| | | TTCAAAAACGGTAAAGTTACCAAATCTTACTCTTTC |
| | | GACGAAATCCGTAAAAACGCTCAGCTGAACATCGAA |
| | | CTGGAAGCTGCTCACCACTAA |
| SEQ ID NO: 26 | Example of nucleotide | AACAAACGTTCTAAAAAAGGTCGTCTGTCTCTGCAC |
| | sequence of | CGTACCCCGGCTGGTAACTTCGTTACCCTGGAAGAA |
| | hNAMPTcif-T2 | GGTAAAGGTGACCTGGAAGAATACGGTCAGGACCTG |
| | | CTGCACACCGTTTTCAAAAACGGTAAAGTTACCAAA |
| | | TCTTACTCTTTCGACGAAATCCGTAAAAACGCTCAG |
| | | CTGAACATCGAACTGGAAGCTGCTCACCACTAA |
| SEQ ID NO: 27 | Example of nucleotide | CTGTCTCTGCACCGTACCCCGGCTGGTAACTTCGTT |
| | sequence of | ACCCTGGAAGAAGGTAAAGGTGACCTGGAAGAATAC |
| | hNAMPTcif-T3 | GGTCAGGACCTGCTGCACACCGTTTTCAAAAACGGT |
| | | AAAGTTACCAAATCTTACTCTTTCGACGAAATCCGT |
| | | AAAAACGCTCAGCTGAACATCGAACTGGAAGCTGCT |
| | | CACCACTAA |
| SEQ ID NO: 28 | Example of nucleotide | CCGGCTGGTAACTTCGTTACCCTGGAAGAAGGTAAA |
| | sequence of | GGTGACCTGGAAGAATACGGTCAGGACCTGCTGCAC |
| | hNAMPTcif-T4 | ACCGTTTTCAAAAACGGTAAAGTTACCAAATCTTAC |
| | | TCTTTCGACGAAATCCGTAAAAACGCTCAGCTGAAC |
| | | ATCGAACTGGAAGCTGCTCACCACTAA |
| SEQ ID NO: 29 | Example of nucleotide | TGGCGCGAACCGAGCTTTATGGCGCTGAGCTCTTAC |
| | sequence of hvWF1- | GTTGTTACCAACGGTCTGGGTATCAACGTTTTCAAA |
| | hNAMPTcif | GACCCGGTTGCTGACCCGAACAAACGTTCTAAAAAA |
| | | GGTCGTCTGTCTCTGCACCGTACCCCGGCTGGTAAC |
| | | TTCGTTACCCTGGAAGAAGGTAAAGGTGACCTGGAA |
| | | GAATACGGTCAGGACCTGCTGCACACCGTTTTCAAA |
| | | AACGGTAAAGTTACCAAATCTTACTCTTTCGACGAA |
| | | ATCCGTAAAAACGCTCAGCTGAACATCGAACTGGAA |
| | | GCTGCTCACCACTAA |
| SEQ ID NO: 30 | Example of nucleotide | TGCAGCCAGCCGCTGGATGTGATTCTGCTGCTGGAT |
| | sequence of hvWF2- | GGCAGCAGCAGCTTTCCGGCGAGCTATTTTGATGAA |
| | hNAMPTcif | ATGAAAAGCTTTGCGAAAGCGTTTATTAGCAAAGCG |
| | | AACATTGGCCCGCGCCTGACCCAGGTGAGCGTGCTG |
| | | CAGTATGGCAGCATTACCACCATTGATGTGCCGTGG |
| | | AACGTGGTGCCGGAAAAAGCGCATCTGCTGAGCCTG |
| | | GTGGATGTGATGCAGCGCGAAGGCGGCCCGAGCCAG |
| | | ATTGGCGATGCGCTGGGCTTTGCGGTGCGCTATCTG |
| | | ACCAGCGAAATGCATGGCGCGCGCCCGGGCGCGAGC |
| | | AAAGCGGTGGTGATTCTGGTGACCGATGTGAGCGTG |
| | | GATAGCGTGGATGCGGCGGCGGATGCGGCGCGCAGC |
| | | AACCGCGTGACCGTGTTTCCGATTGGCATTGGCGAT |
| | | CGCTATGATGCGGCGCAGCTGCGCATTCTGGCGGGC |
| | | CCGGCGGGCGATAGCAACGTGGTGAAACTGCAGCGC |
| | | ATTGAAGATCTGCCGACCATGGTGACCCTGGGCAAC |
| | | AGCTTTCTGCATAAACTGTGCAGCGGCTTTGTGCGC |
| | | ATTTGCACCGGCTCTTACGTTGTTACCAACGGTCTG |
| | | GGTATCAACGTTTTCAAAGACCCGGTTGCTGACCCG |
| | | AACAAACGTTCTAAAAAAGGTCGTCTGTCTCTGCAC |
| | | CGTACCCCGGCTGGTAACTTCGTTACCCTGGAAGAA |
| | | GGTAAAGGTGACCTGGAAGAATACGGTCAGGACCTG |
| | | CTGCACACCGTTTTCAAAAACGGTAAAGTTACCAAA |
| | | TCTTACTCTTTCGACGAAATCCGTAAAAACGCTCAG |
| | | CTGAACATCGAACTGGAAGCTGCTCACCACTAA |
| SEQ ID NO: 31 | hCol-hNAMPTcif | ACCAAAAAAACCCTGCGCACCTCTTACGTTGTTACC |
| | | AACGGTCTGGGTATCAACGTTTTCAAAGACCCGGTT |
| | | GCTGACCCGAACAAACGTTCTAAAAAAGGTCGTCTG |
| | | TCTCTGCACCGTACCCCGGCTGGTAACTTCGTTACC |
| | | CTGGAAGAAGGTAAAGGTGACCTGGAAGAATACGGT |
| | | CAGGACCTGCTGCACACCGTTTTCAAAAACGGTAAA |
| | | GTTACCAAATCTTACTCTTTCGACGAAATCCGTAAA |
| | | AACGCTCAGCTGAACATCGAACTGGAAGCTGCTCAC |
| | | CACTAA |
| SEQ ID NO: 32 | Example nucleotide | TCTTACGTTGTTACCAACGGTCTGGGTATCAACGTT |
| | sequence of hNAMPTcif | TTCAAAGACCCGGTTGCTGACCCGAACAAACGTTCT |
| | | AAAAAAGGTCGTCTGTCTCTGCACCGTACCCCGGCT |
| | | GGTAACTTCGTTACCCTGGAAGAAGGTAAAGGTGAC |
| | | CTGGAAGAATACGGTCAGGACCTGCTGCACACCGTT |
| | | TTCAAAAACGGTAAAGTTACCAAATCTTACTCTTTC |
| | | GACGAAATCCGTAAAAACGCTCAGCTGAACATCGAA |
| | | CTGGAAGCTGCTCACCACTAA |
| SEQ ID NO: 33 | Truncated hNAMPTcif- | NKRSKKGRLSLHRTPAGNFVTLEEGKGDLEEYGQDL |
| | T5 (NAMPT422-471, | LHTVFKNGKVTKS |
| | numbering corresponds | |
| | to full length NAMPT, | |
| | e.g SEQ ID NO: 19) | |
| SEQ ID NO: 34 | Truncated hNAMPTcif- | HHHHHHHHENLYFQGNKRSKKGRLSLHRTPAGNFVT |
| | T5 (NAMPT422-471, | LEEGKGDLEEYGQDLLHTVFKNGKVTKS |
| | numbering corresponds | |
| | to full length NAMPT, | |
| | e.g SEQ ID NO: 19) | |
| | (including HIS tag) | |
| SEQ ID NO: 35 | Example of nucleotide | AACAAACGTTCTAAAAAAGGTCGTCTGTCTCTGCAC |
| | sequence of | CGTACCCCGGCTGGTAACTTCGTTACCCTGGAAGAA |
| | hNAMPTcif-T5 | GGTAAAGGTGACCTGGAAGAATACGGTCAGGACCTG |
| | | CTGCACACCGTTTTCAAAAACGGTAAAGTTACCAAA |
| | | TCTTAC |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The general chemical terms used in the formulae herein have their usual meaning.

The present invention is based on a surprisingly direct and essential role for specific macrophage subsets has been identified in modulating tissue regeneration in vivo, demonstrating that a proportion of wound-attracted macrophages form a transient stem cell niche with resident tissue stem cells and induce their activation. Ablation of this niche-specific macrophage subset leads to a severe reduction in the number of proliferating progenitors present within the injury site, and a consequent regeneration deficit. The term injury herein relates broadly to any externally or internally inflicted or present wound where tissue regeneration is required to replace lost tissue or rebuild or regenerate functional tissue lost through any process such as a disease process, the results of infection or trauma, longevity, poor diet and lack of exercise, etc.

An obligate satellite cell-macrophage niche has been identified in real time and within the wound to instigate efficient skeletal muscle regeneration and injury repair. This demonstrates that a proportion of wound-attracted macrophages form a transient stem cell niche and are pro-myogenic. Ablation of this niche-specific macrophage subset leads to a severe reduction in the number of myogenic progenitors present within the injury site, and a consequent muscle regeneration deficit.

Accordingly, along with their well described ability to modulate pro-inflammatory and anti-inflammatory events, specific macrophage populations also provide a transient stem cell activating niche (the cells are spatially constrained together and interact directly in muscle tissue). The stem cells may be tissue stem cells, such as a muscle stem cell. For example, the stem cells are skeletal muscle stem cells (satellite cells). Other muscle stem cells include heart tissue stem cells or non-striated muscle cells.

Accordingly, macrophage derived factors as detailed herein are proposed for use in modulating stem cell activity, in directly activating quiescent tissue stem cells and in tissue regeneration. One macrophage derived factor described herein is Nicotinamide phosphoribosyltransferase (NAMPT, also known as visfatin and PBEF (pre-B cell enhancing factor). NAMPT is identified by the inventors to be upregulated and produced by injury dwelling macrophages. As described and exemplified herein, specific derivatives of NAMPT, in particular truncated forms of the cif motif and various fusion polypeptides, have been developed that induce muscle stem cell proliferation.

Accordingly, the NAMPT polypeptides and fusion proteins are proposed for use in stimulating would healing and improving the quality of healing in order to promote full restoration of tissue function i.e., productive tissue repair and regeneration.

Reference to polypeptides and fusion proteins includes variants that activate quiescent tissue stem cells, and their derivatives comprising adaptations suitable for production, and clinical or commercial use, known in the art, such as enhanced tissue delivery or enhanced signalling functionalities. The term includes orthologs and isoforms.

Reference to “regeneration” in relation to a muscle is used herein in a broad context and includes the flow on effects on muscle and muscle associated tissue as a direct result of muscle stem cell (also called satellite cell) activation. Thus, regeneration includes muscle wound repair and muscle maintenance, growth, repair, augmentation of the ability of muscle cells to productively proliferate and form functional tissues. The term includes generation of muscle tissue, and repair of an injured muscle, and pertains to the process of muscle regeneration (myogenesis) commencing with activation and proliferation of muscle stem cells, proliferation of myoblasts, early differentiation into myocytes and terminal differentiation into myofibres. In one embodiment, regeneration is associated with minimal fibrosis which allows for establishment of native structures or regenerated tissue having normal or approximating normal biological properties rather than fibrotic or weakened tissue. Muscle functional properties may be determined by standard tests of contractile muscle function, including tests for strength (for example eccentric muscle contraction), power and endurance, as well as physical length and volume. The term also includes growth of muscle tissue in commercial cultures.

In certain embodiments, treatment of muscle with C-terminal fragment of NAMPT described herein was associated with little or minimal fibrosis in a clinically relevant volumetric wound model.

The present invention finds particular application in the treatment of, or muscle tissue regeneration in subject having, myopathies where inflammation is particularly undesirable. For example, the invention provides a method of stimulating muscle tissue regeneration in a subject where inflammation is undesirable, the method comprising administering to a muscle an effective amount of a polypeptide, fusion protein, composition or cell as described herein, wherein the a polypeptide, fusion protein, composition or cell as described herein, thereby stimulating muscle tissue regeneration in this subject.

In one embodiment, the inflammation that is undesirable is inflammation mediated by TLR activation, preferably TLR4 activation.

In one embodiment, the subject may be diagnosed with an inflammatory myopathy. Exemplary inflammatory myopathies include polymyositis, dermatomyositis, inclusion body myositis, necrotizing autoimmune myopathy. Other inflammatory myopathies, their clinical features and methods of diagnosing them are described in Dalakas, 2015, N Engl J Med 2015;372:1734-47.

The present invention provides a method of treating an inflammatory myopathy in a subject, the method comprising administering to a muscle of the subject an effective amount of a polypeptide, fusion protein, composition or cell as described herein, thereby treating an inflammatory myopathy. Preferably, the inflammatory myopathy is polymyositis, dermatomyositis, inclusion body myositis or necrotizing autoimmune myopathy.

In one embodiment, the application enables a pharmaceutical or physiologically active regenerative composition comprising one or two or three or four or five of
a polypeptide, fusion protein, nucleic acid, vector, or cell as described herein,
a satellite cell or precursor therefore or progeny thereof
a macrophage or a precursor therefore or progeny thereof
a scaffold or retentive material
a tissue delivery enhancing component.

In a particular embodiment, as described elsewhere herein, the a polypeptide or fusion protein in monomeric or dimeric form is modified to make the agent suitable for attachment to a biological carrier or to the extracellular matrix. In addition, the agent is modified to enhance signalling through the CCR5 receptor by addition of moieties that bind co-receptors such as heparin sulphate proteoglycans (such as syndecans).

In one embodiment, a polypeptide, fusion protein, nucleic acid, vector, cell or composition described herein are for use, or for use in manufacturing compositions for use, in stimulating muscle regeneration in vitro, ex vivo or in vitro.

In one embodiment, the compositions described herein are for use or when used in artificial muscle production (such as fish, bird or other non-human animal muscle for direct or indirect consumption). For example, supplementation to growth media enables scalability and more efficient muscle proliferation.

In one embodiment, the compositions described herein are for use or when used in stem cell therapy. Thus, the compositions support expansion in vitro and/or are included in a transplant (or as a pre-treatment) to promote in vivo expansion and tissue integration.

In one embodiment, the present application provides a method of stimulating tissue regeneration, the method comprising administering to an isolated or tissue-resident tissue stem cell or a precursor thereof an effective amount of a composition comprising or encoding a polypeptide or fusion protein described herein, and optionally a component that enhances delivery to the tissue, wherein the polypeptide or fusion protein binds to tissue stem cells or their precursors and stimulates (activates) quiescent tissue stem cell proliferation and tissue regeneration. In one embodiment, the polypeptide or fusion protein comprises a component or moiety that enhances delivery to the target tissue.

In one embodiment, the compositions described herein are for use or when used, or for use in manufacturing compositions for use, in treating a muscular, neuromuscular, or musculoskeletal deficiency, disorder or injury. Muscular, neuromuscular, or musculoskeletal deficiencies, disorders or injuries are known in the art. Deficiencies and disorders are found, for example, and without limitation in sarcopenia, cachexia and the muscular dystrophies, muscle atrophy, muscle pseudo hypertrophy or muscle dystrophy conditions and myopathies. All appropriate formats such as Swiss-style use, method of treatment and/or EPC 2000 style claims are encompassed.

The term “isolated cell” as used herein refers to a cell that has been removed from an organism in which it was originally found or a descendant of such a cell. The cell may have been cultured in vitro, e.g., in the presence of other cells. Also, the cell may be destined to later be introduced into a second organism or re-introduced into the organism from which it (or the cell from which it is descended) was isolated.

The term “isolated population of cells” or the like, refers to a population of cells that has been removed and separated from a mixed or heterogeneous population of cells. In some embodiments, an isolated population is a substantially pure population of cells as compared to the heterogeneous population from which the cells were isolated or enriched.

In one embodiment, the application enables a pharmaceutical or physiologically active regenerative composition comprising one or two or three or four or five of:
(i) a polypeptide, fusion protein, nucleic acid, vector, or cell as defined herein,
(ii) a tissue stem cell (such as a satellite cell) or precursor therefore or progeny thereof
(iii) a macrophage or a precursor therefore or progeny thereof
(iv) a scaffold or retentive material
(v) a tissue delivery enhancing component.

In one embodiment, the present application provides cellular compositions comprising one or more of one or more stem cells, stromal cells, pre-satellite cells or satellite cells, pre-macrophages or macrophages or macrophage derived factors as described herein. In one embodiment, multipotent “tissue stem cell” include a pre-muscle cell or any pre-macrophage cells from which these cell may be produced in an essentially native form or modified to express heterologous or autologous factors. Similarly, the term multipotent “tissue stem cell” may include activated progeny of the tissue stem cell.

Tissue stem cells including muscle stem cells can be isolated (for ex vivo or in vitro or in vivo procedures) or induced.

A stem cell can be contacted with a media or composition comprising a a polypeptide, fusion protein, nucleic acid, vector, or cell for any amount of time. For example, a stem cell can be contacted with a polypeptide, fusion protein, nucleic acid, vector, or cell for 12 hours, 1 day, 2 days, 3 days, 4 days, 5 days, 6 days, 1 week or more. The stem cell can be induced or stimulated to differentiate into a cell lineage selected from the group consisting of mesoderm, endoderm, ectoderm, neuronal, mesenchymal, and hematopoietic lineage.

In some embodiments, the stem cell is a human stem cell, a multipotent adult stem cell, a pluripotent adult stem cell or an embryonic stem cell.

Human adult stem cells are mitotic and typically one daughter cell remains a stem cell. Adult tissue comprises one or more resident committed progenitor or stem cells that occupy a specific niche in their tissue and actively sense and respond to their local environment. Each tissue typically has its own resident committed stem cell committed to producing progeny that differentiate into a specific range of cell types. Muscle tissue comprises satellite cells to are committed to producing myoblasts. Other well studied stem cells of this type are mesenchymal stem cells (MSC) that produce many different cell types inter alia muscle, cartilage, bone, fat, and haematopoietic stem cells (HSC) that produce all blood cells and the haematopoietic system, and neural stem cells (NSC). All tissue contains resident stem cell populations, including heart, gut and liver. Adult stem cells are typically multipotent which refers to a cell that is able to differentiate into some but not all of the cells derived from all three germ layers. Thus, a multipotent cell is a partially differentiated cell. MSC, for example can be obtained by a number of methods well known in the art. See U.S. Pat. Nos. 5,486,358; 6,387,367; and U.S. Pat. No. 7,592, 174, and USPN 2003/0211602. MSC may be derived from bone, fat and other tissues where they reside. “Derived” from does not refer to direct derivation and merely indices where they were originally derived.

In one embodiment, the stem cell is a non-embryonic or adult multipotent stem cell.

In one embodiment the stem cell is a HSC or MSC.

Adult stem cell expressing CCR5 can be stimulated to undergo differentiation by exposure to a polypeptide, fusion protein, nucleic acid, vector, or cell described herein. Cells are monitored to changes in expression of for example myogenic regulatory factors known in the art.

Cells may be cultured in standard media or specifically defined media.

Cell expression may be modified by techniques know in the art.

An induced or partially induced pluripotent stem cell is a convenient source of stem cells. These are derivable from a differentiated adult cell, such as human foreskin cells.

Human iPS cells can be generated by introducing specific sets of reprogramming factors into a non-pluripotent cell which can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. For example, the reprograming factors can be introduced into the cells using one or more plasmids, lentiviral vectors, or retroviral vectors. In some cases, the vectors integrate into the genome and can be removed after reprogramming is complete. In some cases, the vectors do not integrate (e.g., those based on a positive-strand, single-stranded RNA species derived from non-infectious (non-packaging) self-replicating Venezuelan equine encephalitis (VEE) virus, Simplicon RNA Reprogramming Kit, Millipore, SCR549 and SCR550). The Simplicon RNA replicon is a synthetic in vitro transcribed RNA expressing all four reprogramming factors (OKG-iG; Oct4, Klf4, Sox2, and Glis1) in a polycystronic transcript that is able to self-replicate for a limited number of cell divisions. Human induced pluripotent stem cells produced using the Simplicon kit are referred to as “integration-free” and “footprint-free.” Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells express alkaline phosphatase, SOX-2, OCT-4, Nanog and Tra-1-60 markers.

The terms “naive” and “primed” identify different pluripotency states of human iPS cells. Characteristics of naive and primed iPS cells are described in the art. Naive human iPS cells exhibit a pluripotency state similar to that of ES cells of the inner cell mass of a pre-implantation embryo. Such naive cells are not primed for lineage specification and commitment. Female naive iPS cells are characterized by two active X chromosomes. In culture, self-renewal of naive human iPS cells is dependent on leukemia inhibitory factor (LIF) and other inhibitors. Cultured naive human iPS cells display a clonal morphology characterized by rounded dome-shaped colonies and a lack of apico-basal polarity. Cultured naive cells can further display one or more pluripotency makers as described elsewhere herein. Under appropriate conditions, the doubling time of naive human iPS cells in culture can be between 16 and 24 hours.

Primed human iPSC express a pluripotency state similar to that of post-implantation epiblast cells. Such cells are primed for lineage specification and commitment. Female primed iPSCs are characterized by one active X chromosome and one inactive X chromosome. In culture, self-renewal of primed human iPSCs is dependent on factors such as fibroblast growth factor (FGF) and activin. Cultured primed human iPSCs display a clonal morphology characterized by an epithelial monolayer and display apico-basal polarity. Under appropriate conditions, the doubling time of primed human iPSCs in culture can be 24 hours or more depending upon the level from the adult cells from which they were derived.

Embryonic stem cells (ESC) are characteristically pluripotent i.e., they have the capacity, under different conditions, to differentiate to cell types characteristic of all three germ cell layers (endoderm, mesoderm and ectoderm). Pluripotent cells are characterized primarily by their ability to differentiate to all three germ layers. In some embodiments, a pluripotent cell is an undifferentiated cell. Pluripotent cells also have the potential to divide in vitro for more than one year or more than 30 passages.

ESC are typically the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see U.S. Pat. Nos. 5,945,577, 5,994,619, 6,235,970). Exemplary distinguishing embryonic stem cell characteristics include, without limitation, gene expression profile, proliferative capacity, differentiation capacity, karyotype, responsiveness to particular culture conditions.

In one embodiment the stem cell is adult.

In one embodiment the stem cells are autologous or heterologous to the subject.

In one embodiment, the stem cell is mammalian or human.

Macrophages and stem cells including satellite cells may be prepared using art recognised methods and as described herein and include the use of iPSC and optionally gene editing procedures.

One embodiment isolated macrophages or stem-cell derived macrophages are modified to express a polypeptide or fusion protein described herein. Generally, M2 type macrophages are selected or provided.

In one embodiment stem cells are contacted with a polypeptide, fusion protein, nucleic acid, vector, or cell in vitro, ex vivo or in vivo as described herein to induce activation and proliferation. Stem cells treated in vitro or ex vivo may be introduced into a wound site to effect repair or administered systemically to effect regeneration of damaged tissue or to treat or improve muscle related conditions as described herein.

A polypeptide, fusion protein, nucleic acid, vector, or cell expressing said polypeptide or fusion protein may be administered in the form of functionalized hydrogels either alone or together with cells for transplantation. Such hydrogels or similar biomaterials or scaffolds provide enhanced transplantation efficiency at the wound site.

Hydrogels may be ECM based such as fibrin based. Alternatively, hydrogels may be non-ECM based such as acrylamide based using RAFT technology (see Chiefari et al Macromol. 31:5559-5526, 1998 and Fairbanks et al Advanced Drug Delivery Reviews 91: 141-152, 2015). Suitable materials regulate release kinetics, and have desired mechanical and physical properties for tissue regeneration as known in the art.

In one embodiment, satellite cells are encapsulated in CCR5 functionalised hydrogels or other biomaterials.

Kits comprising the cellular compositions and/or agents described herein are also provided. Kits suitable for muscle repair or regeneration are specifically contemplated. The polypeptide, fusion protein, nucleic acid, vector, or cell can be pre-formulated for administration or ingredients for formulation can be provided with the kit. The polypeptide, fusion protein, nucleic acid, vector, or cell is for example formulated in a hydrogel or other supporting vehicle for topical application. The polypeptide, fusion protein, nucleic acid, vector, or cell may be, for example, lyophilised or liquid.

The terms “protein,” “polypeptide,” and “peptide,” used interchangeably herein, include polymeric forms of amino acids of any length unless that length is defined, including coded and non-coded amino acids and chemically or biochemically modified or derivatized amino acids. The terms also include polymers that have been modified, such as polypeptides having modified peptide backbones.

Proteins are said to have an “N-terminus’ be “N-terminal” and to have a “C-terminus” or be “C-terminal.” The term “N-terminus” relates to the start of a protein or polypeptide, terminated by an amino acid with a free amine group (—NH2). The term “C-terminus” relates to the end of an amino acid chain (protein or polypeptide), which is in nature terminated by a free carboxyl group (—COOH). In the present application reference to C-terminal and N-terminal fragments broadly describe the region of the full length molecule from which the elected part is derived and it excludes a full length or a native molecule. A C-terminal fragments does not have to but may include all the C-terminal amino acids and N-terminal fragments do not have to but may include all the N-terminal amino acids.

The application discloses and enables the use of a range of polypeptides, fusion proteins, nucleic acids, vectors, or cells based upon the initial findings described in the examples.

A number of peptide modifications are known in the art to stabilise peptides against serum proteases or to promote intracellular positioning and these are encompassed. Some such modified peptides can be expressed from nucleic acids in a cell, others are manufactured synthetically. Similarly, where it is desirable to target the polypeptide or fusion protein described herein to one or more specific cell types, this may be achieved either by ex vivo manipulation of target cells, or incorporation of targeting moieties able to bind specifically to target cells or tissues such as ECM binding moieties, as known in the art.

A “conservative amino acid substitution” is one in which the naturally or non-naturally occurring amino acid residue is replaced with a naturally or non-naturally occurring amino acid residue having a similar side chain. Families of amino acid residues having similar side chains have been defined in the art. These families include amino acids with basic side chains (e.g., Lys, Arg, His), acidic side chains (e.g., Asp, Glu), uncharged polar side chains (e.g., Gly, Asn, Gln, Ser, Thr, Tyr, Cys), nonpolar side chains (e.g., Ala, Val, Leu, Ile, Pro, Phe, Met, Trp), beta-branched side chains (e.g., Thr, Val, Ile) and aromatic side chains (e.g., Phe, Trp, His). Thus, a predicted nonessential amino acid residue in a CCR5, for example, may be replaced with another amino acid residue from the same side chain family. Other examples of acceptable substitutions are substitutions based on isosteric considerations (e.g. norleucine for methionine) or other properties (e.g. 2-thienylalanine for phenylalanine). A full amino acid sub-classification is set out in Table 2 and exemplary substitutions are set out in Table 3.

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, Threonine |
| Polar/large | Asparagine, Glutamine |
| Hydrophobic | Tyrosine, Valine, Isoleucine, Leucine, Methionine, Phenylalanine, |
| | Tryptophan |
| Aromatic | Tryptophan, Tyrosine, Phenylalanine |
| Residues that influence chain | Glycine and Proline |
| orientation | |

TABLE 3
Exemplary and Preferred Amino Acid Substitutions

| | | Preferred |
|---|---|---|
| Original residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

In some embodiments Trp residues are substituted.

A polypeptide or fusion protein described herein may comprise modifications known to modify the pharmacokinetic features of peptides, such as by increasing protease resistance in vivo.

In one embodiment, the peptide comprises one or more of a linker or spacer such as GGS or repeats of GGS and variants known in the art), a modified or non-natural or non-proteogenic amino acid, a modified side-chain, a modified backbone, terminal modified groups or comprises a modified spatial constraint or is a D-retro-inverso peptide. In one embodiment, the peptide is a pseudopeptide, peptoid, azapeptide, cyclized, stapled, ether or lactam peptide or comprises a spatial constraint.

In one embodiment, the polypeptide or fusion protein described herein is conjugated or otherwise attached/bound/expressed with as appropriate to a lipid, carbohydrate, polymer, protein, nanoparticle, peptide, proteoglycan, antibody or fragment or antigen binding form thereof, aptamer, or nucleic acid.

In one embodiment, the polypeptide or fusion protein described herein specifically binds to muscle cells or muscle cell tissue or associated structures eg, ECM.

In one embodiment, polypeptide or fusion protein described herein includes physiologically or pharmaceutically acceptable salts, hydrates, sterioisomers, and pro-drugs.

In one embodiment, non-essential amino acids may be altered. Reference to “non-essential” amino acid residue means a residue that can be altered from the wild-type sequence of a polypeptide without abolishing or substantially altering its ability to bind to an endogenous or heterologous CCR5.

In one embodiment, the polypeptide or fusion protein described herein comprises or encodes an amino acid sequence having 1, 2, 3, 4, 5 or 6 conservative (for example those outlined in Table 3 above) or non-conservative amino acid substitution, deletion or addition to the above sequences but retains CCR5 interacting activity.

In one embodiment, the invention also provides a nucleic acid molecule from which the polypeptide or fusion protein described herein is expressible. Nucleotide sequences encoding polypeptides or fusion proteins described herein are herein disclosed.

In one embodiment, the nucleic acid molecule is an RNA or DNA or RNA: DNA or a chemically modified form thereof.

In one embodiment, a proportion of at least one type of nucleotide (e.g, cysteine and/or uracil), is chemically modified to increase its stability in vivo.

In one embodiment, the nucleic acid is in the form of a viral or non-viral vector.

In one embodiment, the polypeptide fusion protein, nucleic acid, vector, cell or composition described herein is administered to cells ex vivo. The present invention encompasses the use of genetically modified cell depots (e.g. CAR T-cells, TCRs, genetically modified macrophage, etc).

In one embodiment, the polypeptide or fusion protein comprises an antibody or antibody fragment that targets the agent specifically to target cells, such as muscle stem cells.

In one embodiment, the present application provides a pharmaceutical or physiological composition comprising a polypeptide, fusion protein, nucleic acid, vector or cell as defined herein above.

The application enables a method of treating a muscle injury or a person with a diminished or suboptimal ability to repair or regenerate muscle, comprising administering to the subject an effective amount of a composition comprising a polypeptide, fusion protein, nucleic acid, vector or cell, or composition comprising a polypeptide, fusion protein, nucleic acid, vector or cell, sufficient to stimulate muscle stem cell proliferation and muscle regeneration.

Compositions include physiologically or pharmacologically or pharmaceutically acceptable vehicles that are not biologically or otherwise undesirable. Pharmacologically acceptable salts, esters, pro-drugs, or derivatives of a compound described here is a salt, ester, pro-drug, or derivative that is not biologically or otherwise undesirable.

In some embodiments, the polypeptide or fusion protein is modified. Polypeptide and fusion protein activity are tolerant to additional moieties, flanking residues and substitutions within the defined boundaries. Similarly backbone modifications and replacements, side-chain modifications and N and C-terminal modifications are conventional in the art. Generally, the modification is to enhance stability or pharmacological profile, targeting/delivery. For example, peptide cyclisation or stapling is conventional for enhancing peptide stability. In another embodiment, peptides or agents are in the form of micro or nano-particles or bubbles, gels, liposomes, conjugates or fusion proteins comprising moieties adapted for stability, delivery or specificity to the target tissue.

In one embodiment, agents or their encoding nucleic acids where appropriate are assembled in liposomes, hydrogels, emulsions, viral vectors, viral-like particles or virosomes.

In one embodiment, specific binding moieties such as antibody or antibody fragments or mimics are used to target polypeptide or fusion proteins to the muscle environment.

In one embodiment, polypeptide or fusion proteins are delivered through biological synthesis in vivo such as via delivery of mRNA, gene editing such as CRISPR components, or bacteria or cells.

Compositions generally comprise a polypeptide or fusion protein, peptidomimetic or an encoding nucleic acid where appropriate, and a pharmaceutically acceptable carrier and/or diluent. In one embodiment, the carrier may be a nanocarrier.

In one embodiment, the polypeptides or fusion proteins of the present disclosure are not naturally occurring molecules, but instead are modified forms of naturally occurring molecules which do not possess certain features or functions of the naturally occurring full length molecules. For example, NAMPT enzymatic activity may be absent.

In another embodiment, the polypeptide or fusion protein described herein is constrained by means of a linker which is covalently bound to at least two amino acids in the peptide. Various cyclisation strategies are known in the art to increase stability and cellular permeability.

In some embodiments, the polypeptide or fusion protein described herein is delivered in the form of nucleic acid molecules encoding same or pro-drugs thereof or vectors comprising nucleic acid molecules encoding same or pro-drugs thereof. In one embodiment, the nucleic acid is mRNA. The polypeptide or fusion protein described herein may bind the surface of the muscle stem cell or function internally to stimulate signalling and proliferation.

In another embodiment, the polypeptide fusion protein, nucleic acid, vector or cell described herein is combined, in addition to other pharmaceutically acceptable carriers, with amphipathic agents such as lipids which exist in aggregates as micelles, insoluble monolayers, liquid crystals or lamellar layers in aqueous solution.

In one embodiment, the disclosure enables a composition comprising a polypeptide or fusion protein as described herein which interacts with endogenous CCR5 proteins for use as a medicament or for use in therapy.

In another aspect, the present disclosure enables a composition for stimulating muscle stem cell proliferation comprising a polypeptide or fusion protein as described herein or a nucleic acid molecule from which the peptide is expressible.

In one embodiment, the subject composition is co-administered with a second physiologically active, therapeutic or prophylactic or regenerative agent. Illustrative cytokines include without limitation one or more of IGF-1, TGF-β, GDF-5, bFGF, PDGF-b3, IL-4.

In another aspect, the present disclosure provides for the use of the polypeptide, fusion protein, nucleic acid, vector or cell as described herein in the manufacture of a medicament for stimulating muscle regeneration or in stem cell therapy.

In one embodiment, the application provides screening assays for CCR5-interacting agents as described herein, comprising assessing the ability of agents to induce muscle stem cell proliferation and muscle generation or indicators thereof.

Peptide-based therapeutics provide useful molecules because they are known to be potent and selective against biological targets that are otherwise difficult to manipulate with small molecules. To improve the pharmacokinetic properties of linear peptides, modified peptides have been successfully developed.

The peptides of the present disclosure comprise amino acids. Reference to “amino acid” includes naturally occurring amino acids or non-naturally occurring amino acids.

Peptide compounds are generally and conventionally modifiable by addition of moieties, flanking peptide residues, and substitutions within understood parameters. Peptides can furthermore comprise routine modified backbones, side chains, peptide bond replacements, and terminal modifications using standard peptide chemistries.

The amino acids incorporated into the amino acid sequence described herein may be L-amino acids, D-amino acids, L-β-homo amino acids, D-β-homo amino acids or N-methylated amino acids, sugar amino acids, and/or mixtures thereof. Non-natural amino acids may not be recognised by proteases and may therefore alter the half-life. In one embodiment, the D-retro inversion sequence is employed.

Non-naturally occurring amino acids include chemical analogues of a corresponding naturally occurring amino acid. Examples of unnatural amino acids and derivatives include, but are not limited to, 4-amino butyric acid, 6-aminohexanoic acid, 4-amino-3-hydroxy-5-phenylpentanoic acid, 4-amino-3-hydroxy-6-methylheptanoic acid, t-butylglycine, nor leucine, norvaline, phenylglycine, ornithine, sarcosine, 2-thienyl alanine and/or D-isomers of amino acids.

In one embodiment, peptides are modified to enhance their pharmacodynamics properties using art recognised modifications. Peptides may be substituted, such as alanine substituted, or substituted with cross linkable moieties and/or linked. Suitable residues may comprise additional alpha-carbon substitutions selected from hetero-lower alkyl, hetero-methyl, ethyl, propyl and butyl. Peptide bond replacements such as trifluoroethylamines are used to produce more stable and active peptidomimetics.

Accordingly, cyclic or stapled peptides, peptoids, peptomers, and peptidomimetic forms of peptides are encompassed.

Backbone constrained peptidomimetics and cyclic peptides are protected against exopeptidases. Peptides can be cyclised coupling N-to C-terminus after cleavage. This can be achieved by direct coupling or by introduction of specific functional groups that permit defined cyclization by a biorthogonal reaction. Illustrative modifications Cys-Cys disulphide bridges, inclusion of sidechain modifications to include linkers forming macro lactam peptides, thio ether peptides or stapled peptides etc. Click variants are particularly useful for peptide cyclization. Another approach uses 2-amino-d,1-dodecanoic acid (Laa) couples to the N-terminus and by replacing Asn with the lipoamine.

A more defined structure can be obtained by use of a more rigid back bone with heterocycles, N-methylated amine bonds or methylated alpha-carbon atoms.

Among the techniques used for peptide stapling, the two-component double Cu-catalysed azide-alkyne cycloaddition (CuAAC) strategy constrains the peptides in the bioactive conformation and simultaneously improves pharmacokinetic properties. Moreover, this strategy uses unnatural azido amino acids that can be easily synthesised and facilitates the functionalisation of the staple, fluorescent-labelled tags and photo-switchable linkers. The independent functionalisation of the staple can be particularly useful as the complex functionality is added to the staple rather than the N- or C-terminus of the peptide. In addition, this approach only requires one linear peptide to generate a variety of functionalised stapled peptides, facilitating the exploration of various functionalities on the linker and thus properties of the overall peptide.

Azapeptides are peptide analogs in which one or more of the amino residues is replaced by a semicarbazide. This substitution of a nitrogen for the a-carbon center results in conformational restrictions, which bend the peptide about the aza-amino acid residue away from a linear geometry. The resulting azapeptide turn conformations have been observed by x-ray crystallography and spectroscopy, as well as predicted based on computational models. In biologically active peptide analogs, the aza-substitution has led to enhanced activity and selectivity as well as improved properties, such as prolonged duration of action and metabolic stability.

Half-life may also be increased by acylating or amidating ends. Peptoids are produced with N-alklyated oligoglycines side chains. In some embodiments, peptides may be acetylated, acylated (e.g., lipopeptides), formylated, amidated, phosphorylated (on Ser, Thr and/or Tyr), sulphated or glycosylated.

The term “macrocyclization reagent” or “macrocycle-forming reagent” as used herein refers to any reagent which may be used to prepare a peptidomimetic macrocycle by mediating the reaction between two reactive groups. Reactive groups may be, for example, an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents may additionally include, for example, Ru reagents known in the art such as Cp·RuCl(PPh₃)₂, [Cp·RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbine complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Acc. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

In one embodiment, a peptidomimetic macrocycle exhibits improved biological properties such as increased structural stability, increased affinity for a target, increased resistance to proteolytic degradation when compared to a corresponding non-macrocyclic polypeptide. In another embodiment, a peptidomimetic macrocycle comprises one or more α-helices in aqueous solutions and/or exhibits an increased degree of α-helicity in comparison to a corresponding non-macrocyclic polypeptide.

For example, the sequence of the peptide can be analyzed and azide-containing and alkyne-containing amino acid analogs of the invention can be substituted at the appropriate positions. The appropriate positions are determined by ascertaining which molecular surface(s) of the secondary structure is (are) required for biological activity and, therefore, across which other surface(s) the macrocycle forming linkers of the invention can form a macrocycle without sterically blocking the surface(s) required for biological activity. Such determinations are made using methods such as X-ray crystallography of complexes between the secondary structure and a natural binding partner to visualize residues (and surfaces) critical for activity; by sequential mutagenesis of residues in the secondary structure to functionally identify residues (and surfaces) critical for activity; or by other methods. By such determinations, the appropriate amino acids are substituted with the amino acids analogs and macrocycle-forming linkers of the invention. For example, for a helical secondary structure, one surface of the helix (e.g., a molecular surface extending longitudinally along the axis of the helix and radially 45-135° degree. about the axis of the helix) may be required to make contact with another biomolecule in vivo or in vitro for biological activity. In such a case, a macrocycle-forming linker is designed to link two carbons of the helix while extending longitudinally along the surface of the helix in the portion of that surface not directly required for activity.

The peptidomimetic macrocycle may comprise a helix in aqueous solution. For example, the peptidomimetic macrocycle may exhibit increased helical structure in aqueous solution compared to a corresponding non-macrocyclic polypeptide. In some embodiments, the peptidomimetic macrocycle exhibits increased thermal stability compared to a corresponding non-macrocyclic polypeptide. In other embodiments, the peptidomimetic macrocycle exhibits increased biological activity compared to a corresponding non-macrocyclic polypeptide. In still other embodiments, the peptidomimetic macrocycle exhibits increased resistance to proteolytic degradation compared to a corresponding non-macrocyclic polypeptide. In yet other embodiments, the peptidomimetic macrocycle exhibits increased ability to penetrate living cells compared to a corresponding non-macrocyclic polypeptide.

The term “amino acid analog” refers to a molecule which is structurally similar to a naturally occurring amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group or for the substitution of the amino or carboxy group by a similarly reactive group (e.g., substitution of the primary amine with a secondary or tertiary amine, or substitution or the carboxy group with an ester).

The peptide may comprise an N-terminal acetyl, formyl, myristoyl, palmitoyl, carboxyl, 2-furosyl and or a C-terminal hydroxyl, amide, ester or thioester group. In one embodiment, the peptide is acetylated at the N-terminus and amidated at the C-terminus. In one embodiment, chelators are introduced for example DOTA, DPTA. Peptides may be modified by, for example pegylation, lipidation, xtenylation, pasylation and other approaches to extend the half-life of the peptide in vivo or in vitro. In one embodiment, pegylation is used to increase peptide solubility and bioavailability. Various forms of peg are known in the art and include HiPeg, branched and forked Peg, releasable Peg, heterobifunctional Peg with end group NHS esters, malaimeide, vinyl sulphone, pyridyl disulphide, amines and carboxylic acids. Examples of therapeutic pegylated peptides include pegfilagrastin (Neulasta) made Amgen.

Linkers or spacers may be amino acids or nucleic acids or other atomic structures known in the art, typically between 2 and 10 amino acids or nucleotides in length. Spacers should be flexible enough to allow correct orientation of CCR5-interacting constructs as described herein, such as those including nanoparticles, antibody fragments, liposomes, cell penetrating and/or intracellular delivery moieties. One form of spacer is the hinge region from IgG suitable for use when the construct comprises an antigen binding moiety for cellular targeting.

Antigen-binding molecules include for example extracellular receptors, antibodies or antibody fragments (including molecules such as an ScFv). Signal peptides may be present at the N-terminal end. Bispecific antibodies capable of selectively binding to two or more epitopes are known in the art and could be used in the present CCR5 interacting agents to bind for example to the muscle environment or other substrate.

In one embodiment, the peptide is conjugated or otherwise associated (covalent or non-covalent attachment) with a delivery agent. In one embodiment the delivery agent delivers the peptide to tissue, a target cell or cell population.

Derivatives of polypeptides or fusion proteins include biologically active fragments thereof as described herein comprising the structures described or orthologs. Systematic shortening or alanine scanning or modelling around the conserved motif can be routinely conducted to identify minimal peptides with CCR5 agonist effect.

Derivatives also include molecules having a percent amino acid or polynucleotide sequence identity over a window of comparison after optimal alignment. In one embodiment the percentage identity is at least 80%-99% including any number in between 80 and 99.

Suitable assays for the biological activity of peptides or agents are known to the skilled addressee and are described in the specification.

In some embodiment, markers of peptide activity include upregulation of satellite cell signalling (eg, MAPK), stem cell and myoblast proliferation and differentiation.

In one embodiment, the polypeptide or fusion protein is modified with a moiety which is not a naturally occurring amino acid residue. The moiety may be selected from the group consisting of a detectable label, a non-naturally occurring amino acid as described herein, a reactive group, a fatty acid, cholesterol, a lipid, a bioactive carbohydrate, a nanoparticle, a small molecule drug, and a polynucleotide. In one particular embodiment, the moiety is a detectable tag label. In one example the detectable label is selected from the group consisting of a fluorophore, a fluorogenic substrate, a luminogenic substrate, and a biotin. Art recognised tags or labels include affinity agents and moieties for detection include fluorescent and luminescent compounds, metals, dyes. Other useful moieties include affinity tags, biotin, lectins, chelators, lanthanides, fluorescent dyes, FRET acceptor/donors.

In one embodiment, the polypeptide or fusion protein which may comprise a detectable label, is accompanied in a kit with a modified control version of the agent wherein the conserved residues of the polypeptide or fusion protein are substituted with for example alanine. Kits comprising the agents are proposed for sale and may be used for screening purposes or therapeutic purposes.

Peptides of this type may be obtained through the application of recombinant nucleic acid techniques as, for example, described in Sambrook et al. MOLECULAR CLONING. A LABORATORY MANUAL (Cold Spring Harbour Press, 1989), in particular Sections 16 and 17; Ausubel et al CURRENT PROTOCOLS IN MOLECULAR BIOLOGY (John Wiley & Sons, Inc. 1994-1998), in particular Chapters 10 and 16; and Coligan et al. CURRENT PROTOCOLS IN PROTEIN SCIENCE (John Wiley & Sons, Inc. 1995-1997), in particular Chapters 1, 5 and 6.

Alternatively, peptides of this type may be synthesised using conventional liquid or increasingly solid phase synthesis techniques. For example, initial reference may be made to solution synthesis or solid phase synthesis as described, for example, by Atherton and Sheppard in SOLID PHASE PEPTIDE SYNTHESIS: A PRACTICAL APPROACH (IRL Press at Oxford University, Oxford, England, 1989), see particularly Chapter 9, or by Roberge et al. (1995 Science 269:202).

Azapeptide synthesis was previously hampered by tedious solution-phase synthetic routes for selective hydrazine functionalization. Recently, the submonomer procedure for azapeptide synthesis, has enabled addition of diverse side chains onto a common semicarbazone intermediate, providing a means to construct azapeptide libraries by solution- and solid-phase chemistry. In brief, aza residues are introduced into the peptide chain using the submonomer strategy by semicarbazone incorporation, deprotonation, N-alkylation, and orthogonal deprotection. Amino acylation of the resulting semicarbazide and elongation gives the desired azapeptide. Furthermore, a number of chemical transformations have taken advantage of the orthogonal chemistry of semicarbazone residues (e.g., Michael additions and N-arylations). In addition, oxidation of aza-glycine residues has afforded azopeptides that react in pericyclic reactions (e.g., Diels-Alder and Alder-ene chemistry). The bulk of these transformations of aza-glycine residues have been developed by the Lubell laboratory, which has applied such chemistry in the synthesis of ligands with promising biological activity for treating diseases such as cancer and age-related macular degeneration. Azapeptide analogues of growth hormone-releasing peptide-6 (His-d-Trp-Ala-Trp-d-Phe-Lys-NH2, GHRP-6) have for example been pursued as ligands of the cluster of differentiation 36 receptor (CD36) and show promising activity for the development of treatments for angiogenesis-related diseases, such as age-related macular degeneration, as well as for atherosclerosis. Azapeptides have also been employed to make a series of conformationally constrained second mitochondria-derived activator of caspase (Smac) mimetics that exhibit promising apoptosis-inducing activity in cancer cells. The synthesis of cyclic azapeptide derivatives was used to make an aza scan to study the conformation-activity relationships of the anticancer agent cilengitide, cyclo(RGDf-N(Me)V), and its parent counterpart cyclo(RGDfV), which exhibit potency against human tumor metastasis and tumor-induced angiogenesis. Innovations in the synthesis and application of azapeptides are described in Acc Chem Res. 2017 Jul. 18;50(7):1541-1556.

Alternatively, peptides can be produced by digestion of an adaptor polypeptide with proteinases such as endoLys-C, endoArg-C, endoGlu-C and staphylococcus V8-protease. The digested fragments can be purified by, for example, high performance liquid chromatographic (HPLC) techniques. Measures that may be taken to optimize pharmacodynamics parameters of peptides and peptide analogs are described by Werle M. et al (2006) Strategies to improve plasma half-life time of peptide and protein drugs amino Acids 30(4):351-367; and Di L (2014) Strategic approaches to optimising peptide ADME properties AAPS J 1-10.

The polypeptide or fusion protein may be stabilised for example via nanoparticles, liposomes, micelles or for example PEG as known in the art. Methods to form liposomes are described in: Prescott, Ed. Methods in Cell Biology, Volume XIV, Academic Press, New York, N.Y. (1976), p. 33 et seq., the contents of which is incorporated herein by reference. Polymer nanoparticles ideally use surfactants that are not toxic or physically adsorbed to the nanoparticle. In one aspect, biodegradable surfmers are used. For example, biodegradable, poly(ethylene glycol) (PEG) ylated N-(2-hydroxypropyl) methacrylamide (HPMA) based surfmers are synthesized and used to stabilize lipophilic NPs. In particular, the NP core is made from a macromonomer comprising a poly(lactic acid) (PLA) chain functionalized with HPMA double bond. The nanoparticle forming polymer chains are then constituted by a uniform poly(HPMA) backbone that is biocompatible and water soluble and hydrolysable PEG and PLA pendants assuring the complete degradability of the polymer. The stability provided by the synthesized surfmers is studied in the cases of both emulsion free radical polymerization and solution free radical polymerization followed by the flash nanoprecipitation of the obtained amphiphilic copolymers.

Other stabilising or heterologous moieties include NMEG, ECM binding, syndecan binding albumin, albumin binding proteins, immunoglobulin Fc domain.

Traditional Fc fusion proteins and antibodies are examples of unguided interaction pairs, whereas a variety of engineered Fc domains have been designed as asymmetric interaction pairs as described by Spiess et al. (2015) Molecular Immunology 67(2A): 95-106. Fc conjugates may comprise an amino acid sequence that is derived from an Fc domain of an IgG (IgG1, IgG2, IgG3, or IgG4), IgA (IgA1 or IgA2), IgE, or IgM immunoglobulin. Such immunoglobulin domains may comprise one or more amino acid modifications (e.g., deletions, additions, and/or substitutions) that promote hetero or homo dimeric or multimeric amyloid formation within the host cell.

In some embodiments, nanoparticles comprising the polypeptide, fusion protein, nucleic acid, vector or cell can be further modified by the conjugation of tissue type specific binding agents, antibodies or fragments thereof known in the art.

Other suitable binding agents are known in the art and include antigen binding constructs such as affimers, aptamers, or suitable ligands (receptors) or parts thereof.

Antibodies, such as monoclonal antibodies, or derivatives or analogs thereof, include without limitation: Fv fragments; single chain Fv (scFv) fragments; Fab′ fragments; F(ab′)2 fragments; humanized antibodies and antibody fragments; camelized antibodies and antibody fragments, and multivalent versions of the foregoing. Multivalent binding reagents also may be used, as appropriate, including without limitation: monospecific or bispecific antibodies; such as disulfide stabilized Fv fragments, scFv tandems (scFv) fragments, diabodies, tribodies or tetrabodies, which typically are covalently linked or otherwise stabilized (i.e. leucine zipper or helix stabilized) scFv fragments.

The term “antibody fragments”, as used herein, include any portion of an antibody that retains the ability to bind to the epitope recognized by the full length antibody. Examples of antibody fragments include, but are not limited to, Fab, Fab′ and F(ab′)₂, Fd, single-chain Fvs (scFv), disulfide-linked Fvs (dsFv), and fragments comprising either a V_{L} or V_{H} region. Antigen-binding fragments of antibodies can comprise the variable region(s) alone or in combination with a portion of the hinge region, CH1, CH2, CH3, or a combination thereof. Preferably, the antibody fragments contain all six CDRs of the whole antibody, although fragments containing fewer than all six CDRs may also be functional.

“Single-chain FVs” (“scFvs”) are antigen-binding fragments that contain the heavy chain variable region (V_{H}) of an antibody linked to the light chain variable region (V_{L}) of the antibody in a single polypeptide, but lack some or all of the constant domains of the antibody. The linkage between the V_{H} and V_{L} can be achieved through a short, flexible peptide selected to assure that the proper three-dimensional folding of the V_{L} and V_{H} regions occurs to maintain the target molecule binding-specificity of the whole antibody from which the scFv is derived. scFvs lack some or all of the constant domains of antibodies.

Methods of making receptor-specific binding agents generally, particularly based on natural ligands, but including antibodies and their derivatives and analogs and aptamers, are known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of target-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, New Jersey and the pSKAN Phagemid Display System, commercially available from MoBiTec, LLC of Marco Island, Florida. Aptamer technology is described for example and without limitation in U.S. Pat. Nos. 5,270,163; 5,475,096; 5,840,867 and 6,544,776.

Optionally, one or more modified amino acid residues are selected from the group consisting of: a glycosylated amino acid, a PEGylated amino acid, a farnesylated amino acid, an acetylated amino acid, a biotinylated amino acid, and an amino acid conjugated to a lipid moiety, and an amino acid conjugated to an organic derivatizing agent. CCR5 interacting peptides may comprise at least one N-linked sugar, and may include two, three or more N-linked sugars. Peptides may also comprise O-linked sugars. CCR5 interacting peptides or agents may be produced in a variety of cell lines that glycosylate the protein in a manner that is suitable for patient use, including engineered insect or yeast cells, and mammalian cells such as COS cells, CHO cells, HEK cells and NSO cells. In some embodiments the CCR5 peptide is glycosylated and has a glycosylation pattern obtainable from a Chinese hamster ovary cell line. In most embodiments the CCR5 interacting agent is synthesised and component parts added using techniques known in the art.

In some embodiments, the subject polypeptide or fusion proteins described herein have a half-life of about 0.5, 1, 2, 3, 4, 6, 12, 24, 36, 48, or 72 hours in a mammal (e.g., a mouse or a human). Alternatively, they may exhibit a half-life of about 0.5, 1, 2, 3, 4, 5, 6, 8, 10, 12, 14, 20, 25, or 30 days in a mammal (e.g., a mouse or a human) depending upon conjugate and carrier features and the mode of administration. In some embodiments, peptides are modified to maximise retention in the muscle tissue and to avoid or minimise systemic circulation. Agents may be administered in a range of retention enhancing compositions known in the art, such as gels, foams, glues, hydrogels, patches, and films, and the like.

The size of peptide may be modified to alter its hydrodynamic radium and renal clearance. PEGylation and lipidation often with linkers are established modifications to increase serum half life of agents by reducing clearance and protection from proteases. Second-generation PEGylation processes introduced the use of branched structures as well as alternative chemistries for PEG attachment. In particular, PEGs with cysteine reactive groups such as maleimide or iodoacetamide allow the targeting of the PEGylation to a single residue within a peptide reducing the heterogeneity of the final product. Furthermore, biodegradable hydrophilic amino acid polymers that are functional analogs of PEG have been developed, including XTEN (see US 20190083577) and PAS that are homogeneous and readily produced. Chemical linkage of antibody to peptide as developed by ConX illustrate a range of hybrid peptide half-life extension methods that promise to overcome may of the disadvantages of earlier methods.

Oral and injectable solution solubilizing excipients include water-soluble organic solvents (polyethylene glycol 300, polyethylene glycol 400, ethanol, propylene glycol, glycerin, N-methyl-2-pyrrolidone, dimethylacetamide, and dimethylsulfoxide), non-ionic surfactants (Cremophor EL, Cremophor RH 40, Cremophor RH 60, d-α-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750), water-insoluble lipids (castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil), organic liquids/semi-solids (beeswax, d-α-tocopherol, oleic acid, medium-chain mono- and diglycerides), various cyclodextrins (α-cyclodextrin, β-cyclodextrin, hydroxypropyl-β-cyclodextrin, and sulfobutylether-β-cyclodextrin), and phospholipids (hydrogenated soy phosphatidylcholine, distearoylphosphatidylglycerol, l-α-dimyristoylphosphatidylcholine, l-α-dimyristoylphosphatidylglycerol). The chemical techniques to solubilize agents for oral and injection administration include pH adjustment, cosolvents, complexation, microemulsions, self-emulsifying drug delivery systems, micelles, liposomes, and emulsions.

### Constructs/Vectors

A construct or vector for expressing a polypeptide or fusion protein described herein from a recipient cell can comprise one or more DNA regions comprising a promoter operably linked to a nucleotide sequence encoding the peptide. The promoter can be inducible or constitutive. Examples of suitable constitutive promoters include, e.g., an immediate early cytomegalovirus (CMV) promoter, an Elongation Growth Factor-Ia (EF-Ia) gene promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Examples of inducible promoters include, but are not limited to a metallothionine promoter, a glucocorticoid promoter, a progesterone promoter, and a tetracycline promoter.

The expression constructs may be generated by any suitable method including recombinant or synthetic techniques, utilizing a range of vectors known and available in the art such as plasmids, bacteriophage, baculovirus, mammalian virus, artificial chromosomes, among others. The expression constructs can be circular or linear, and should be suitable for replication and integration into eukaryotes. Viruses, which are useful as vectors include, but are not limited to, retroviruses, adenoviruses, adeno-associated viruses, herpes viruses and lentiviruses. A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to the subject stem cells. A number of retroviral systems are known in the art.

In a specific embodiment of the present invention, where the peptide is provided as a nucleic acid encoding the peptide, the nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide or other intracellular targeting moiety. Alternatively, a nucleic acid can be introduced intracellularly and incorporated within host cell DNA for expression.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” may be used and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A nucleic acid molecule as described herein may in any form such as DNA or RNA, including in vitro transcribed RNA or synthetic RNA. Nucleic acids include genomic DNA, cDNA, mRNA, recombinantly produced and chemically synthesized molecules and modified forms thereof. A nucleic acid molecule may be single stranded or double stranded and linear or closed covalently to form a circle. The RNA may be modified by stabilizing sequences, capping, and polyadenylation. RNA or DNA and may be delivered as plasmids to express the peptide. RNA-based approaches are routinely available.

The term “RNA” relates to a molecule which comprises ribonucleotide residues and preferably being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

An optimised mRNA based composition could comprise a 5′ and 3′ non translated region (5′-UTR, 3′-UTR) that optimises translation efficiency and intracellular stability as known in the art. In one embodiment, removal of uncapped 5 ‘-triphosphates can be achieved by treating RNA with a phosphatase. RNA may have modified ribonucleotides in order to increase its stability and/or decrease cytotoxicity. For example, in one embodiment, in the RNA, 5-methylcytidine is substituted partially or completely, for cytidine. In one embodiment, the term “modification” relates to providing an RNA with a 5’-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap. The term “5′-cap” includes a 5′-cap analog that resembles the RNA cap structure and is modified to possess the ability to stabilize RNA and/or enhance translation of RNA. Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

A further modification of RNA may be an extension or truncation of the naturally occurring poly(A) tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alphal-globin, beta-globin. RNA having an unmasked poly-A sequence is translated more efficiently than RNA having a masked poly-A sequence. In order to increase stability and/or expression of the RNA it may be modified so as to be present in conjunction with a poly-A sequence, preferably having a length of 10 to 500, more preferably 30 to 300, even more preferably 65 to 200 and especially 100 to 150 adenosine residues. In order to increase expression of the RNA it may be modified within the coding region so as to increase the GC-content to increase mRNA stability and to perform a codon optimization and, thus, enhance translation in cells. Modified mRNA may be synthesised enzymatically and packaged into nanoparticles such as lipid nanoparticles and administered, for example intramuscularly.

The nucleic acid molecule can be entrapped in microcapsules prepared, for example, by coacervation techniques or by interfacial polymerization, in colloidal drug delivery systems (e.g., liposomes, microspheres, microemulsions, nanoparticles and nanocapsules), or in macroemulsions. Such techniques are known in the art and disclosed in Remington, the Science and Practice of Pharmacy, 20^{th} Edition, Remington, J., ed. (2000). Targeted delivery of agents to particular cell subsets can enhance the therapeutic index. Antibody targeted agents that bind to cells comprising an antigen recognized by the antibody or binding fragments thereof. This include for example maleimide functionalized PEG-PLGA polymeric nanoparticles, or simply combining the CCR5 interacting polypeptide or fusion protein in a composition comprising a delivery moiety or shuttle agent.

Ex vivo approaches contemplate the administration of gene editing such as CRISPR components to modify cells to contain or express a polypeptide or fusion protein as described herein.

### Administration

In accordance with this disclosure, the compositions or agents comprising or encoding polypeptide or fusion protein disclosed herein can be administered to patients for would healing or to delay, maintain, or regenerate muscle in various conditions associated with muscle loss or diminished ability to regenerate functionally.

The compositions may be delivered by injection, by topical or mucosal application, by inhalation or via oral route including modified release modes, over periods of time and in amounts which are effective to stimulate muscle regeneration levels in a subject. Administration may be topical or systemic (e.g., parenteral via for example intravenous, intraperitoneal, intradermal, sub cutaneous or intramuscular routes) or targeted. In one embodiment, administration of CCR5-interacting agent is systemic or directly to a wound. Sub cutaneous or intramuscular routes may be directly to an affected muscle tissue.

A polypeptide, fusion protein, nucleic acid, vector or cell described herein can be formulated in the form of ointments, creams, patches, powders, or other formulations suitable for topical formulations. Small molecular weight polypeptide or fusion protein formulations can deliver the agent from skin to deeper muscle tissue. Accordingly, such formulations may comprise one or more agents that enhance penetration of active ingredient through skin. For topical applications, the polypeptide, fusion protein, nucleic acid, or vector can be included in wound dressings and/or skin coating compositions.

The amount of the agent to be administered may be determined by standard clinical techniques by those of average skill within the art. In addition, in vitro assays may optionally be employed to help identify optimal dosage ranges. The precise dose to be employed will also depend on the nature of the agent and other clinical factors (such as the condition of the subject their weight, age, other conditions, the route of administration and type of composition (cellular, scaffolded, hydrogel baes or oral formulations). The precise dosage to be therapeutically or prophylactically effective and non-detrimental can be determined by those skilled in the art. Pharmaceutical compositions are conveniently prepared according to conventional pharmaceutical compounding techniques. See, for example, Remington, the Science and Practice of Pharmacy, 20^{th} Edition, Remington, J., ed. (2000) and later editions.

Reference to an effective amount includes a therapeutically or physiologically or regeneratively effective amount. A “therapeutically-effective amount” as used herein means that amount of the composition comprising chemokine receptor agonist activity which is effective for producing some desired therapeutic effect in at least a sub-population of cells in an animal at a reasonable benefit/risk ratio applicable to any medical treatment. For example, an amount of a polypeptide or fusion protein administered to a subject that is sufficient to produce a statistically significant, measurable muscle repair or regeneration. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. Routes of administration suitable for the instant compositions with vary depending upon its format and include both local and systemic administration. Generally, local administration results in more polypeptide or fusion protein or cell treated with a polypeptide or fusion protein being delivered to a specific location as compared to the entire body of the subject, whereas, systemic administration results in delivery to essentially the entire body of the subject. One method of local administration is by intramuscular injection.

In accordance with the present invention, the term “administering” also include transplantation of a cell into a subject. As used herein, the term “transplantation” refers to the process of implanting or transferring at least one cell into a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). Skilled artisan is well aware of methods for implanting or transplantation of stem cells for muscle repair and regeneration, which are amenable to the present invention. See for example, U.S. Pat. No. 7,592,174 and U.S. Pat. Pub. No. 2005/0249731, content of both of which is herein incorporated by reference.

As described herein regeneration of muscle tissue by the present methods may be associated with minimal fibrosis. Specifically, the methods and agents described herein may reduce and/or inhibit formation of scar-like tissue in the damaged or non-regenerating or atrophying muscle tissue. Accordingly, in some embodiments, formation of scar-like tissue formation in the damaged muscle tissue is reduced by at least 5%, at least 10%, at least 20%, at least 30%, at least 40%, at least 50%, at least 60%, at least 70%, at least 80%, at least 90%, at least 95%, or 100% relative to a control without the present agents. Adipose deposition may be similarly reduced.

However, suitable dosage ranges for intravenous administration of the polypeptide or fusion protein described herein are generally about 1.25-5 micrograms of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral compositions preferably contain 10% to 95% active ingredient.

By “derivative” is meant an agent or active that has been derived from the polypeptide or fusion protein by modification of the amino acid sequence, or, for example by conjugation or complexing or expression (eg, as a fusion protein) with other chemical moieties or by post-translational modification techniques as would be understood in the art. The term “derivative” also includes within its scope alterations that have been made to a parent sequence including additions, or deletions that provide for functionally equivalent or functionally enhanced molecules.

By “isolated” is meant material that is substantially or essentially free from components that normally accompany it in its native state.

The term “subject,” includes patient, and refers to any subject of medical or veterinary interest. Subjects may be a vertebrate subject, such as mammalian subject (e.g, bovines, pigs, dogs, cats, equine, lama, camelids, etc.), non-mammals, reptiles birds, fish. The subject includes a human, for whom prophylaxis or therapy is desired. The subject may be in need of prophylaxis or treatment for a cancer, wound care, sarcopenia or other pathology, disease, disorder or condition associated with tissue degeneration.

The term “polynucleotide” or “nucleic acid” as used herein designates mRNA, RNA, CRNA, cDNA or DNA. The term typically refers to oligonucleotides greater than 30 nucleotides in length.

The term sequence “identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base {e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” may be understood to mean the “match percentage” calculated by the DNASIS computer program (Version 2.5 for Windows; available from Hitachi Software Engineering Co., Ltd., South San Francisco, California, USA) using standard defaults as used in the reference manual accompanying the software. Amino acid sequence identity may also be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term sequence “similarity” refers to the percentage number of amino acids that are identical or constitute conservative amino acid substitutions as defined in Table 3 above. Similarity may be determined using sequence comparison programs such as GAP (Deveraux et al, 1984 Nucleic Acids Research 12:387-395). In this way, sequences of a similar or substantially different length to those cited herein might be compared by insertion of gaps into the alignment, such gaps being determined, for example, by the comparison algorithm used by GAP. Methods involving conventional molecular biology techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., (2001); and Current Protocols in Molecular Biology, ed. Ausubel et al., Greene Publishing and Wiley-Interscience, New York, (1992) (with periodic updates). Immunology techniques are generally known in the art and are described in detail in methodology treatises such as Current Protocols in Immunology, ed. Coligan et al., Greene Publishing and Wiley-Interscience, New York, (1992) (with periodic updates); Advances in Immunology, volume 93, ed. Frederick W. Alt, Academic Press, Burlington, Mass., (2007); Making and Using Antibodies: A Practical Handbook, eds. Gary C. Howard and Matthew R. Kaser, CRC Press, Boca Raton, Fl, (2006); Medical Immunology, 6^{th} ed., edited by Gabriel Virella, Informa Healthcare Press, London, England, (2007); and Harlow and Lane ANTIBODIES: A Laboratory Manual, Second edition Cold Spring Harbor Laboratory Press, Cold Spring Harbor, N.Y., (2014). Conventional methods of gene transfer and gene therapy may also be adapted for use in the present invention. See, e.g., Gene Therapy: Principles and Applications, ed. T. Blankenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010. Amino Acids. 2018 January; 50(1):39-68. doi: 10.1007/s00726-017-2516-0. Epub 2017 Nov. 28.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Materials and Methods

### Mouse Volumetric Muscle Loss Injury and Repair Assessment

Injury: male C57BL/6J mice aged between 10-12 weeks were anesthetised and shaved on the hind left leg. A unilateral incision measuring approximately 1 cm was made exposing the underlying fascia. The left hind limb was extended and exteriorised via the incision site by retracting the surrounding tissue. A 3×4 mm full thickness segment of the rectus femoris muscle was removed. Directly after, the injury site was filled with fibrin hydrogel with or without 200 ng or 500 ng of hrNAMPT (1) (hydrogel components; 40 μl, 8 mg/ml human fibrinogen (FIB3, Enzyme Research Laboratories), 4 U/ml bovine thrombin (T4648, Sigma), 5 mM CaCI2, 17 μg/ml of aprotinin (ab146286, Abcam)) which polymerized in the defect. Then, the soft tissue was closed with stitches.

Histology: 10 days after treatment, animals were sacrificed and the wounds were harvested for histological analysis. The defect site and associated proximal and distal segment of the quadriceps muscle (including the rectus femoris, vastus medialis and vastus lateralis) were excised and embedded. Histological analysis was performed on serial paraffin sections (4 μm sections collected passing the central portion of the wound). Multiple sections were stained with Masson's Trichrome (to detect collagen deposition) and the extent of fibrosis (represented by a blue stain) was measured by histomorphometric analysis using ImageJ software (version 1.51 h, National Institutes of Health, USA). To maintain uniformity between samples, the length of the vastus medialis taken at multiple depths ranging from 1.0 mm-3.0 mm serves as a reference between tissue sections to determine the depth of sectioning. For fibrotic quantification, average muscle fibrosis area at each depth was scored and normalised with the area of the rectus femoris. Total area of muscle is determined by calculating the average area of rectus femoris at each depth.

Immune cell profiling and PAX7+ cell quantification with flow cytometry: 4, 6 or 8 days after treatment with either 0.5 μg of hrNAMPT (1) delivered by fibrin hydrogel or control fibrin hydrogel only, mice were euthanised via CO2 asphyxiation. The defect site and associated proximal and distal segment of the quadricep muscles were isolated and placed into 890 μl of complete RPMI (with 10% FBS and 2 mM Glutamax, Life Technologies). The tissue was minced with surgical scissors and 100 μl of 10 mg/ml Collagenase II (Sigma-Aldrich) and 10 μl of 10 mg/ml DNAse I (Biolabs), while 100 μl of dispase II (10 mg/ml) was added into the digestion for PAX7 acquisition. The mixture was vortexed and incubated at 37° C. for 45 min. The collagenase was then inactivated with 500 μl ice-cold PBS, 5% FBS, 5 mM EDTA. The mixture was strained subsequently through 70 μm and 40 μm filters. The cell suspension was further diluted with 1 ml complete RPMI and centrifuged for 10 min at 300×g. The supernatant was discarded and the pellet was resuspended in 250 μl complete RPMI and aliquoted into wells of a 96-well U bottom plate for antibody staining. The cell solutions were centrifuged, supernatant discarded, and washed with PBS. The cell viability stain used was 100 μl of Zombie Aqua (Biolegend) Live-Dead dye diluted in PBS (1:400 dilution) and incubated for 30 min at 4° C. The cells were then blocked with FcX (anti-CD16/32 antibodies, Biolegend, 1 μg/ml) flow cytometry buffer (PBS, 5% FBS). The cells were kept for 20 min at 4° C., washed with flow cytometry buffer and centrifuged. Primary surface antibody staining was done in 2 separate stains with 100 μl of anti-mouse antibody cocktail (Biolegend) diluted in flow cytometry buffer: T cell stain with 2 μg/ml of anti-CD4 (clone RM4.5, #100516), anti-CD8 (clone 53-6.7, #100738), and anti-CD3 (clone 17A2, #100220. Neutrophil and macrophage stain with 2 μg/ml of anti-CD11b (clone M1/70, #101208), 1 μg/ml anti-Ly6G (clone 1A8, #127628), 4 μg/ml anti-F4/80 (clone BM8, #123147), 10 μg/ml anti-CD80 (clone 16-10A1, #104714), and 2.6 μg/ml anti-CD206 (clone C068C2, #141720). Cells were stained for 30 min on ice and washed as described above. For internal Foxp3 staining in the T cell panel, cells were fixed with 100 μl fixation/permeabilisation solution (42080, Biolegend) for 35 min. Then cells were washed and resuspended in 100 μl of flow cytometry buffer with 0.5% Saponin and 5 μg/ml anti-Foxp3 (clone 3G3, #35-5773-U100) for 45 min. Cells were then resuspended in flow cytometry buffer (100 μl) and acquired on the Fortessa x20 (Beckman Coulter). Satellite cell flow cytometry staining was performed with 200 μl of antibody cocktail (Biolegend) diluted in flow cytometry buffer: 5 μg/ml of anti-VCAM/CD106 biotin (clone 429 (MVCAM.A), #105703), 2.5 μg/ml of anti-streptavidin (#405250), 2 g/ml of anti-CD45 (clone 30-F11, #103114), anti-CD11b (clone M1/70, #101208), anti-Ly6G (clone 1A8, #127607), 1 μg/ml anti-CD31 (clone MEC13.3, #102507). Cells were stained for 45 min on ice and washed as described above. Cells were also stained with 200 μl flow cytometry buffer with 0.5% saponin with intracellular antibody cocktail: Biolegend 1 ug/ml anti-Ki67 (clone 16A8, #652411), NovusBiologicals 10 μg/ml Anti-Pax7 (clone Pax7/497, #NBP2-34706AF488) for 1 h on ice. Cells were then resuspended in flow cytometry buffer (275 μl) with 25 μl of Invitrogen Count Bright Absolute Counting Beads (25,000 beads, #C36950) and acquired on the Fortessa x20 (Beckman Coulter). All events were acquired and the number of PAX7+ cell per 10,000 wound cells was calculated using the following formula: PAX7+ number in injury=10,000×PAX7+ cell count/[(1/25,000)×beads count×(live cell percentage/100)×total cell number count after digestion]. The same calculation was done to quantify the number of proliferative PAX7+ cells, utilizing cell count of double positive for PAX7 and Ki67.

Immunofluorescence for frozen sections: immunostaining was performed on 10 μm cryosections using standard protocol with antigen retrieval (10 mM Sodium Citrate, 0.05% Tween 20, pH 6.0). Sections were blocked with 2% BSA, 5% Normal Goat Serum in PBS with 0.3% Triton-X and AffiniPure Fab Fragment Goat Anti-Mouse IgG (H+L) (Jackson Immuno Research Laboratories) to minimise the unspecific binding of a mouse antibody on mouse tissue. Antibodies: mouse anti-mouse Pax7 (2 μg/ml, Developmental Studies Hybridoma Bank) and secondary Alexa Fluor-coupled antibodies (Thermo Fisher). Muscle sarcolemma were visualised by Rhodamine-labelled wheat germ agglutinin (WGA) (Vector Laboratories) and nuclei were visualised by staining with DAPI (Sigma-Aldrich).

Quantification of centrally nucleated muscle fibres: Haematoxylin and Eosin (H&E) staining was performed on 4 μm paraffin embedded sections. The number of nuclear centralisations within a muscle fibre was counted from five serial sections per sample by histomorphometric analysis using ImageJ software (version 1.51h, National Institutes of Health, USA). To maintain uniformity between samples, the length of the vastus medialis taken at multiple depths ranging from 1 to 3 mm serves as a reference between tissue sections to determine the depth of sectioning. For average number of centrally nucleated cell quantification, total nuclear count at each depth was normalised with the area of the rectus femoris.

### NAMPT Competitive Binding to CCR5 (ELISA)

hrNAMPT competitive binding to mrCCR5: ELISA plates (Medium binding, Greiner Bio-One) were coated with 1% BSA or 20 nM of recombinant mouse CCR5 (MyBioSource) in PBS overnight at 4° C. Then, wells were blocked for 1 h at room temperature with 1% BSA in PBS containing 0.05% Tween-20 (PBS-T). Wells were washed 3 times with PBS-T and further incubated with hNAMPTcif at increasing concentration (0 nM to 100 nM) for 1 h in PBS-T with 0.1% BSA containing 100 nM hrNAMPT (1) (Peprotech). Bound hrNAMPT (1) molecules were detected using a biotinylated antibody for NAMPT and HRP-streptavidin (Human PBEF/Visfatin DuoSet ELISA, R&D Systems). Signals obtained on BSA-coated wells were used to remove non-specific binding for each hrNAMPT concentrations to obtain specific binding values. Specific binding data were fitted by non-linear regression with Prism 7 to obtain the half maximal inhibitory concentration (IC50) of hNAMPTnif using A450nm=A450nmMin+(A450nmMax−A450nmMin)/(1+10\hat{ }(X-LogIC50)).

### Cell Culture

The mouse muscle cell line C2C12 (Yaffe, D. & Saxel, O. Serial passaging and differentiation of myogenic cells isolated from dystrophic mouse muscle. Nature 270, 725 (1977) were cultured in growth media (Dulbecco's Modified Eagle Medium (4.5 g/l D-Glucose, No L Glutamine, No Sodium Pyruvate (Gibco))+20% Fetal Bovine Solution-One Shot (Gibco)+1% Glut Max 100×(Gibco)). Cells were maintained at 37° C., 5% CO2. Cells at 70% confluence, passage 8 were extracted from T75 flasks with 0.025% Tryspin EDTA (Gibco), neutralised in growth media, spun at 180×g for 5 min to pellet cells. The cells were then resuspended in 10 ml of fresh growth media. 500 μl of cells were plated on a 8-well on cover glass II (Sarstedt) chamber slide at a density of 1×103 cells/ml. Cells were left 4 h at 37° C. to re-attach. For drug treatments, the media were supplemented with appropriate doses and cultured for 6 h.

For isolation of primary mouse myoblasts, limb skeletal muscle from E17.5 C57/BL6J mice were minced and digested in 0.125% Trypsin at 37° C. for 20 min. Fibroblasts were depleted by plating cells in 10 cm2 tissue culture dishes (2 embryos per dish) in proliferation media (DMEM+20% FBS) for 1 h. Media with non-attached cells was re-plated in gelatin-coated 10 cm2 tissue culture dishes in proliferation media for 24 h. Myoblasts were again depleted for fibroblasts prior to co-culturing on gelatin-coated 48 well plates in DMEM+20% FBS+10% L929-conditioned medium. 100,000 myoblasts were plated with either 7,500 MafB/c-Maf deficient (Maf-DKO) macrophages (Aziz, A., Soucie, E., Sarrazin, S. & Sieweke, M. H. MafB/c-Maf deficiency enables self-renewal of differentiated functional macrophages. Science 326, 867-871 (2009)) or 1,000 3T3 cells per well. For drug treatments, the media were supplemented with appropriate doses and cultured for 24 h.

### Cell Surface CCR5 Receptor Concentration

The mouse muscle cell line C2C12 were cultured as described earlier (see above). The mouse macrophage cell line Raw 264.7 (ATCC) were cultured in growth media (Dulbecco's Modified Eagle Medium+10% FBS). Cells were dislodged at 70-80% confluence using a cell scraper and membrane proteins were isolated using an extraction kit (Plasma Membrane Protein Extraction Kit, abcam). CCR5 concentration in the membrane extract was then measure by ELISA (Mouse Ccr5 ELISA Kit, Biorbyt) and the amount of CCR5 per cell was then calculated using [CCR5] (molecules/cell)=[CCR5](ng/cell)/CCR5mw*10\hat{ }−9*N0, where N0=Avogadro constant.

Generating Truncated hNAMPTcif Variants and Proliferative Assays

Human NAMPTcif (hNAMPTcif) and truncated variants of hNAMPTcif were designed by progressively removing N-terminal regions containing positively charged amino acids. Four truncations were produced and numbered T1 to T4. Recombinant protein was produced using a bacterial expression system. hNAMPTcif variants were purified by FPLC using His-tag affinity purification. Cell proliferation assays were performed using C2C12 murine myoblast cells treated with purified recombinant hNAMPTcif variants, as well as full length NAMPT. Cells were treated with 10 nM of recombinant protein for 48 hours in DMEM supplemented with 2% foetal bovine serum at 37° C., 5% CO2. PBS treatment was used as a negative control. 10% foetal bovine serum was used as positive control. Quantification of proliferation was performed using commercially-available CyQuant Proliferation Assay kit (Thermo Fisher Scientific) according to manufacturer's instructions and read using a Synergy H1 plate reader (BioTek). Data were presented as percentage increase versus.

### Generating PIGF2-NAMPTcif Fusion Protein and Proliferative Assays

The heparin-binding sequence of placenta growth factor 2 (PIGF2) was fused to the N-terminus of NAMPTcif and produced using a bacterial expression system. PIGF-NAMPTcif was purified by FPLC using His-tag affinity purification followed by size exclusion chromatography. Cell proliferation assays were performed using C2C12 murine myoblast cells treated with purified recombinant NAMPTcif and full length NAMPT. Cells were treated with 2 nM, 10 nM and 20 nM concentrations of recombinant protein for 48 hours in DMEM supplemented with 2% foetal bovine serum at 37° C., 5% CO2. PBS treatment was used as a negative control. 10% foetal bovine serum was used as positive control. Quantification of proliferation was performed using commercially-available CyQuant Proliferation Assay kit (Thermo Fisher Scientific) according to manufacturer's instructions and read using a Synergy H1 plate reader (BioTek). Data were presented as percentage increase versus negative control. A two-tailed unpaired t-test was used to determine statistical significance.

### TLR4 Signalling Assay

TLR4 receptor activation was assayed using the HEK-Blue TLR4 reporter cell line (InvivoGen). In this cell line, TLR4 and downstream NFκB signalling induces expression and secretion of alkaline phosphatase that can be quantified via colorimetric assay. HEK-Blue cells were treated for 24 hours with 19 nM of NAMPTcif or PIGF-NAMPTcif plus 5 ug/ml polymyxin B to ensure no TLR4 activation by any trace amounts of endotoxin. TLR4 activity was reported as the equivalent amount of activation by bacterial lipopolysaccharides (LPS), thus a standard curve was generated by serial dilutions of LPS from 0.01 to 20 ng/ml. Detection of alkaline phosphatase activity was performed using a QUANTI-Blue kit (InvivoGen) according to manufacturer's instructions and plates were read using a Synergy H1 plate reader (BioTek). Data were presented as equivalent LPS concentration in ng/ml. A one-way ANOVA with Dunnett's multiple comparisons test was performed to determine statistical significance.

### Larval Zebrafish Muscle Injury and EdU Pulse-Chase

Zebrafish larvae (4 dpf) were anaesthetized in 0.01% tricaine (MS-222) (Sigma-Aldrich) in Ringer's solution. Needle-stab injury was carried out in the dorsal myotome, consisting in a single 30-gauge needle puncture that generates an extensive injury with many damaged muscle fibres. Nampt fragment treatments were carried out by incubating 4 dpf needle-stab-injured larvae in 57 nM Nampt in Ringer's solution immediately after injury. Needle-stab-injured larvae at 6 dpf (2 dpi) were transferred into Ringer's solution containing 50 μg ml-1 EdU (Thermo Fisher Scientific) for 1 h and chased for a further 1.5 h before fixation. Samples were developed using the Click-iT EdU Alexa Fluor 647 imaging Kit (Thermo Fisher Scientific) following the manufacturer's protocol, followed by a phalloidin immunostaining (Thermo Fisher Scientific). EdU+ cells in a region encompassing two myotomes on either side of the injury were quantified as the number of EdU+ cells outside the injury region. EdU+ cells in the caudal haematopoietic tissue were excluded from this analysis. Statistical analysis has been conducted using Two-way ANOVA with Tuckey's multiple comparison test.

### Example 2—Exogenous NAMPT Supplementation Accelerates Regeneration in a Mouse Model of Volumetric Muscle Loss

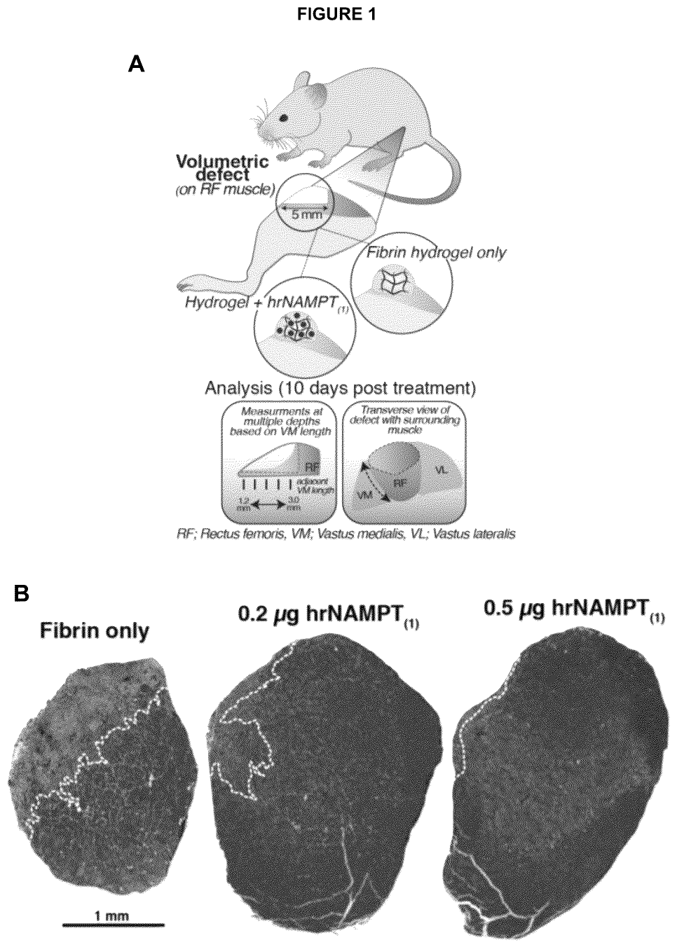

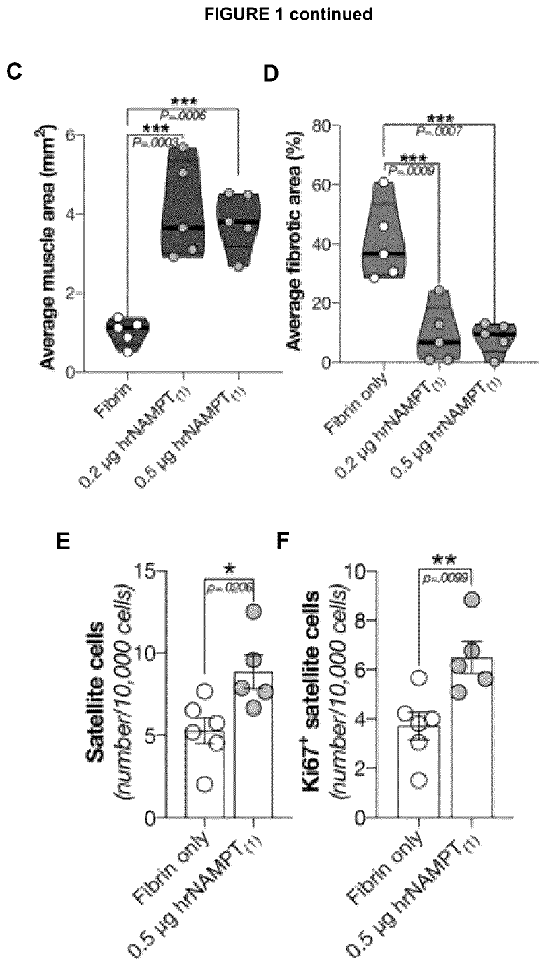

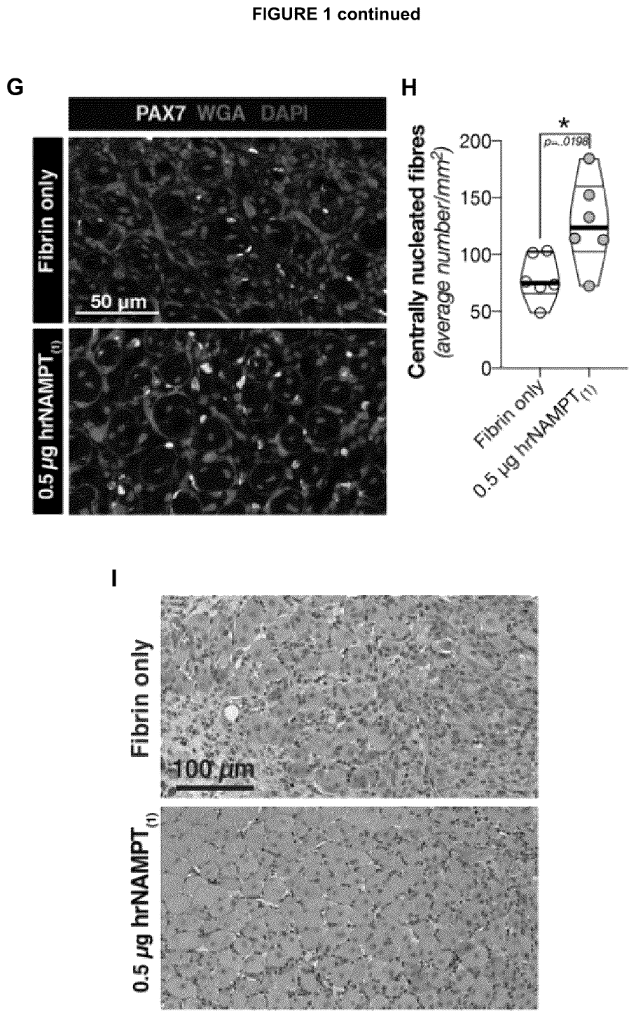

Volumetric muscle loss is an injury paradigm usually refractory to endogenous-stem cell mediated repair processes and is an area of unmet-clinical need. Here it is shown that the addition of exogenously applied NAMPT could accelerate regeneration in a mouse model of volumetric muscle loss. Strikingly, delivery of hrNAMPT into the muscle defect via a fibrin hydrogel, but not a fibrin only control hydrogel, was able to fully restore muscle architecture when applied to the wound site (FIG. 1A-D). On average, treatment with a single dose of hrNAMPT (0.5 μg) at the point of injury led to a 3.276±0.4926 mm² increase in average muscle area and a 34.76±9.32% decrease in average fibrotic area. NAMPT addition in the VML injury model results in a significant increase in both the total number and proportion of proliferating PAX7⁺ satellite cells (FIG. 1E-G) and a significant increase the number of centrally nucleated de novo muscle fibres (FIG. 1H-I).

These findings indicate that exogenously supplied NAMPT protein stimulates muscle repair in the context of an acute injury of adult mammalian muscle.

### Example 3—Selective Signalling of NAMPT Via the CCR5 Receptor is Required to Induce Myoblast Proliferation

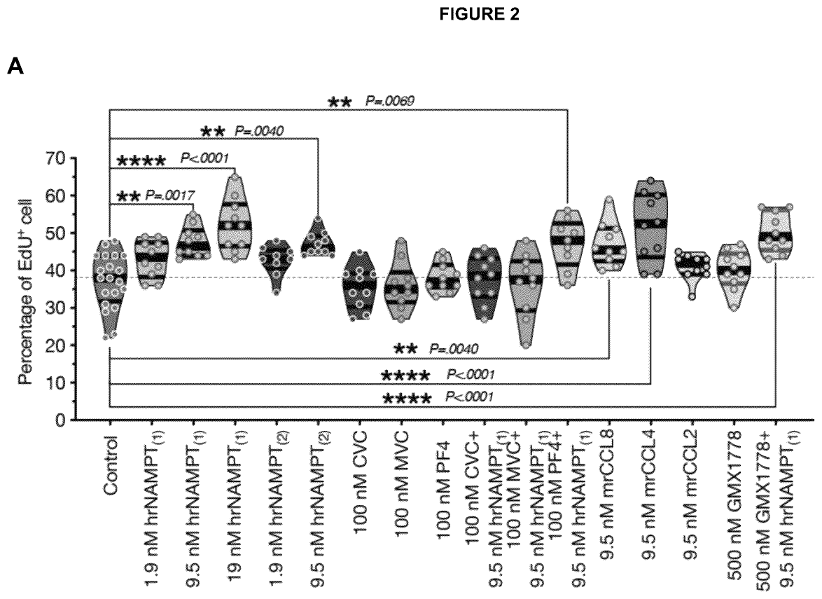

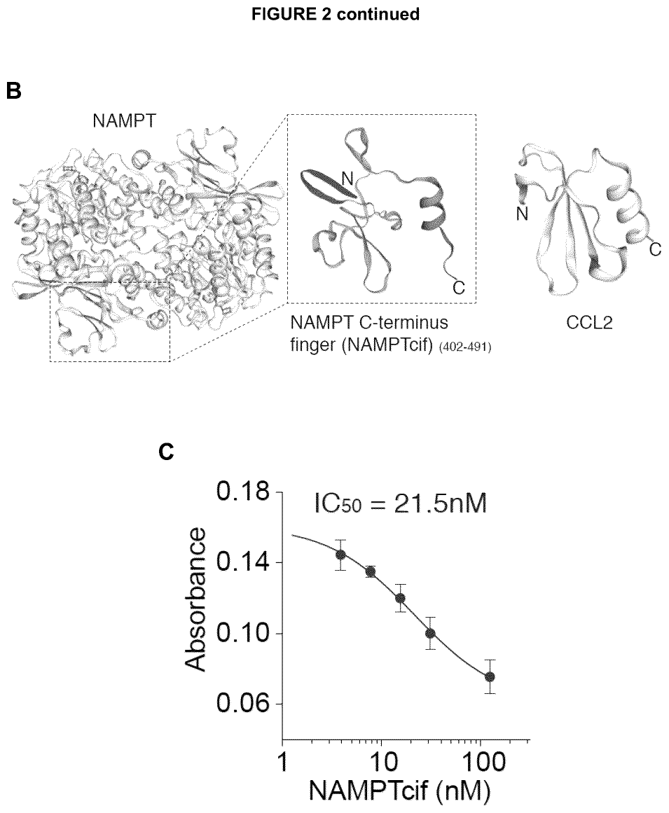

CCR5 receptor is observed at a density 2,470±441 molecules/cell (n=6) on C2C12 myoblasts that is in line with previously documented physiologically relevant levels of CCR5. To determine the physiological relevance of NAMPT-CCR5 interaction, two human recombinant NAMPT protein sources (hrNAMPT(1) and hrNAMPT(2)) were applied to C2C12 myoblasts, and proliferation assayed by means of EdU incorporation. Both sources of NAMPT resulted in comparable and significant dose dependent increases in myoblast proliferation (FIG. 2A). In order to uncouple the intracellular and extracellular roles of NAMPT during proliferation, these myoblasts were treated with GMX1778, a highly specific and potent inhibitor of NAMPT's enzymatic function. Drug treatment had no negative effect on the basal level of C2C12 proliferation in culture and also did not affect the increased myoblast proliferation produced following exogenous NAMPT supplementation (FIG. 2A), highlighting that NAMPT's pro-proliferative role is not reliant on its intracellular enzymatic function. The enhanced proliferative response observed following NAMPT supplementation could be recapitulated in C2C12 cells by the addition of the canonical CCR5 ligands, CCL8/MCP-2 and CCL4/MIP-1β, but not by the CCR2 ligand CCL2/MCP-1 (FIG. 2A). In addition, this proliferative response was blocked in the presence of the dual CCR2/CCR5-antagonist, cenicriviroc (CVC) and CCR5 selective-antagonist maraviroc (MVC), but not in the presence of the CCR2-selective antagonist PF-4136309 (PF) (FIG. 2A).

Collectively, this data highlights that selective signalling of NAMPT via the CCR5 receptor is required to induce myoblast proliferation.

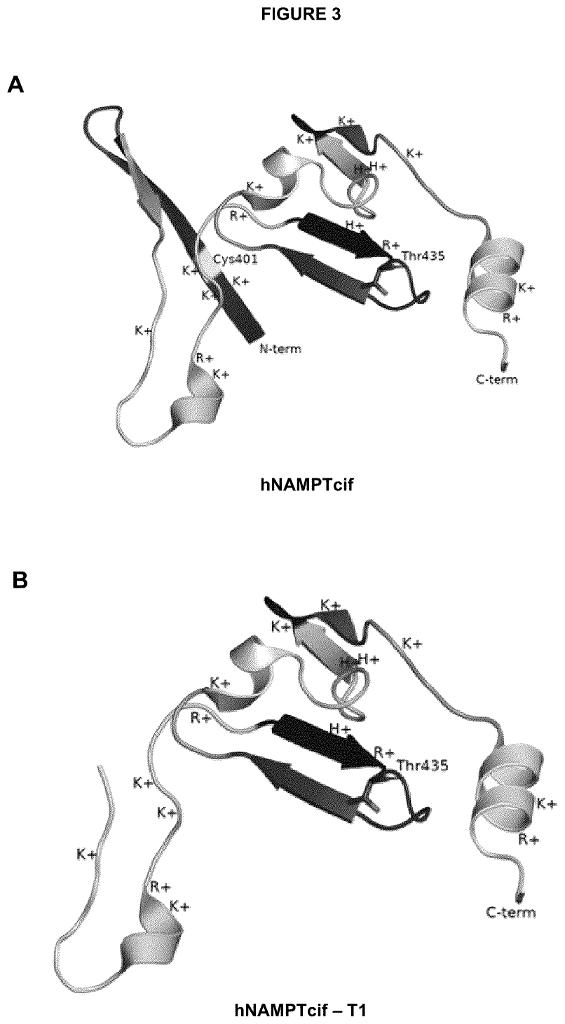

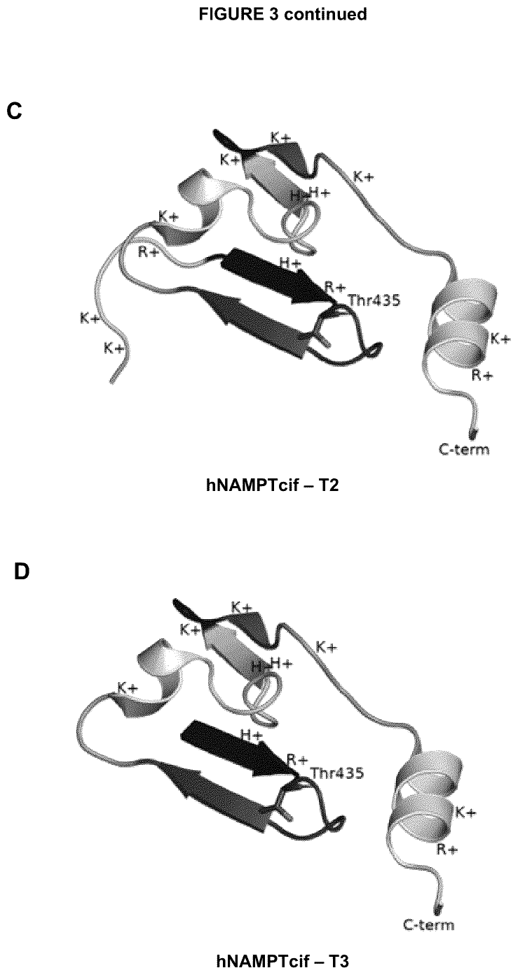

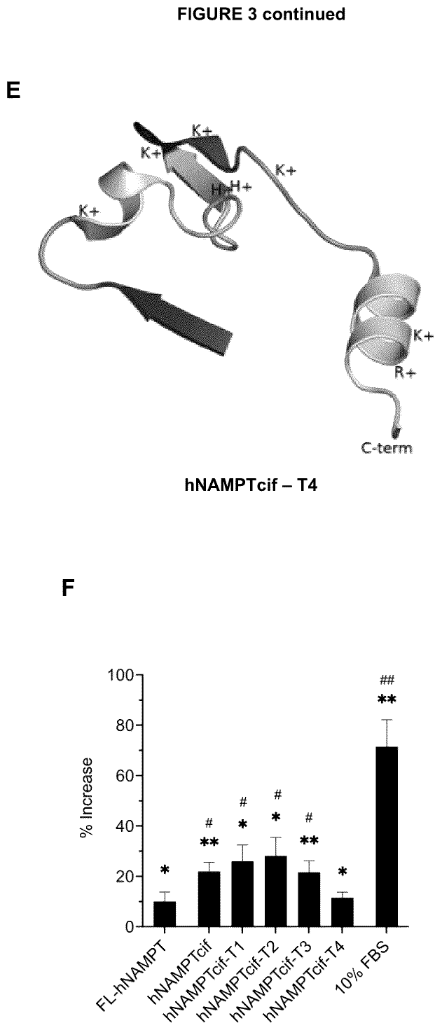

NAMPT, is a large homodimeric intracellular enzyme which acts as cytokine when released in the extracellular milieu. However, the NAMPT domain responsible for cytokine activity is unknown. Re-examining the crystal structure of NAMPT determined that the terminal structure of the NAMPT C-terminus highly resembles classic CCR-binding chemokines (such as CCL2), due to its size and structure (C-terminus α-helix and β-sheets) (FIG. 2B). Moreover, the domain extends out from the core protein structure potentially facilitating receptor binding. Thus, the C-terminus of NAMPT was recombinantly reproduced and its ability to compete with NAMPT binding to CCR5 and to stimulate satellite cell proliferation was tested. Remarkably, this fragment, which is termed herein a “cytokine finger” (cif), inhibits NAMPT binding to CCR5 (IC50=21.5 nM, FIG. 2C) and stimulates satellite cell proliferation in a dose-dependent manner, inducing myoblast proliferation (FIG. 3F).

Collectively, these data demonstrate that the C-terminus cif domain is responsible for the NAMPT's muscle cytokine activity.

### Example 4—Truncated Variants of hNAMPTcif

To further investigate the active fragment of the NAMPT cytokine finger, four truncations of NAMPTcif were produced and numbered T1 to T4 (FIG. 3B-E) and the ability of the variants to stimulate satellite cell proliferation was assessed. Surprisingly, hNAMPTcif and variants T1-T3 demonstrated enhanced stimulated satellite cell proliferation compared to full length hNAMPT (FIG. 3F) despite all molecules containing the cytokine finger.

Collectively, these data unexpectedly demonstrate that NAMPTcif truncations have improved satellite cell proliferation capacity compared to full length NAMPT.

### Example 5—NAMPT Cytokine Derivatives

### ECM-Binding/Syndecan Domain-Fused NAMPTcif

Addition of ECM- and/or syndecan-binding motifs is used as one preferred approach to optimize delivery and increase tonic signalling on CCR5 (see Mochizuki et al Nat. Biomed Engineering 2019). Several proteins bind syndecans such as laminins. One particular syndecan binding moiety is the globular domain of the laminin-α chain having the sequence RKRLQVQLSIRT (SB). Addition of binding molecules may be by synthetic means or using recombinant approaches, as known in the art. Illustrative, non-limiting ECM binding moieties comprises RGD, or YGISR, YIGSR, GFOGER, IKVAV, and GEFYFDLRLKGK.

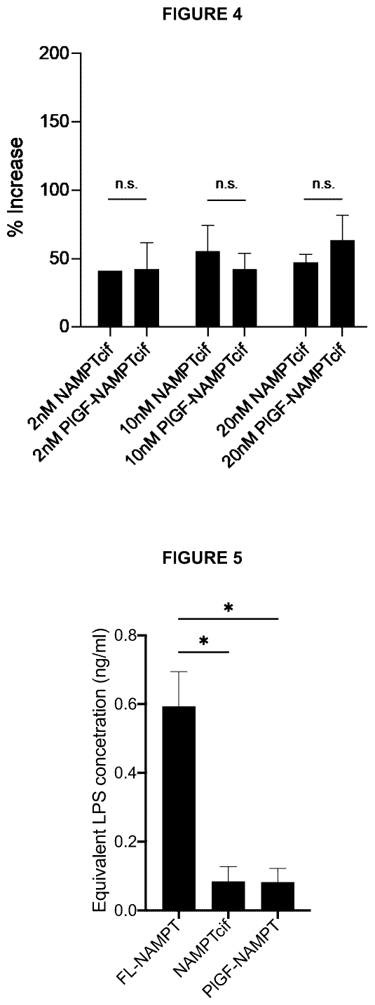

Advantageously, the fusion of the ECM-binding moiety PIGF2 with the N-terminus of NAMPTcif, retained the pro-proliferative activity of unfused NAMPTcif in vitro (FIG. 4). Since there is no ECM to bind in vitro, it is expected that increased activity will be achieved in vivo by the fusion protein, where the ECM-binding moiety PIGF2 will assist in delivery to the cell membrane and increase tonic signalling on CCR5 myoblasts.

### Dimerising the NAMPT Cytokine Finger

NAMPT is naturally a homodimer and known CCR5-binding chemokines are dimers and can form multimers (trimers, tetramers and above through oligomerisation) that modulate receptor binding-affinity and signalling. In one embodiment, NAMPTcif and any polypeptides, fusion proteins or derivatives as described elsewhere in this specification are dimerized. In one approach the single Cysteine residue naturally present at the N-terminus of NAMPTcif is used to force its dimerization. The binding affinity of dimeric NAMPTcif for CCR5 can be tested with ELISA and with surface plasmon resonance (SPR) assays. The activity of dimeric NAMPTcif to promote mouse primary satellite cells proliferation will also indicate muscle generation potential. As an example, quiescent satellite cells are sorted from fresh muscle as used above that relies on negative and positive cell surface markers (CD31−, CD11b−, CD45−, TER119−, Sca1−, CD34+, CD106+) specific for a subset of satellite cells that have been shown to have the highest self-renewal characteristics in vivo. Cells are cultured in vitro and the efficacy of NAMPT derivatives to stimulate proliferation of these primary muscle stem cells assessed.

### Models to Test Different Conditions of Muscle Loss

In addition to the volumetric muscle loss model used in the above examples, there are alternate models that can analyse muscle loss.

As an example, a suitable model for testing function derivatives is an established model of cardiotoxin induced muscle injury. Cardiotoxin is a myonecrotic agent that kills muscle cells without disrupting muscle ECM, providing an important model to test the ECM binding motif containing fusion proteins. It is the most commonly used model in assaying muscle stem cell activation as it leaves the majority of stem cells intact. The standard models include intramuscular injection of cardiotoxin into the Tibialis anterior (TA) muscle and monitoring for restoration of lost fibres. Another model is the mdx mouse, which has also been established at ARMI. The mdx model is used to test both the stem cell activating potential of poylpeptides, fusion proteins and derivatives as well their anti-fibrotic capability, as the mdx model exhibits chronic muscle fibrosis as well as muscle degeneration. Muscle regeneration is tested at established time points using standard histological assays described above.

### Example 6—Reduction of TLR4 Receptor Activation from NAMPTcif Stimulation

In muscle regeneration, pro-inflammatory responses are a cause of fibrosis and scarring during healing. NAMPT has previously been shown to be a modulator of inflammatory programs through binding of inflammatory receptors such as TLR4. Furthermore, activation of TLR4 can induce a hyper inflammatory response leading to severe adverse events, which is problematic for a subset of vulnerable patients, for example patients suffering from an inflammatory myopathy.

Inflammatory myopathies are a group of muscle diseases where the immune system attacks the differentiated muscle cells resulting in muscle loss and chronic inflammation. Hyper inflammatory responses are therefore undesirable and can worsen the patient's condition. There is a need for an effective treatment that can restore muscle in these vulnerable patients, without triggering an adverse inflammatory response.

Here it is confirmed that NAMPT activates TLR4 in vitro, however, advantageously NAMPTcif and PIGF2-NAMPTcif demonstrated abrogated TLR4 activity (FIG. 5). These results suggest that NAMPTcif, polypeptides, fusion proteins and derivatives described herein may improve muscle regeneration by avoiding inflammatory responses.

### Example 7—Human NAMPT Variants Stimulate Muscle Progenitor Proliferation

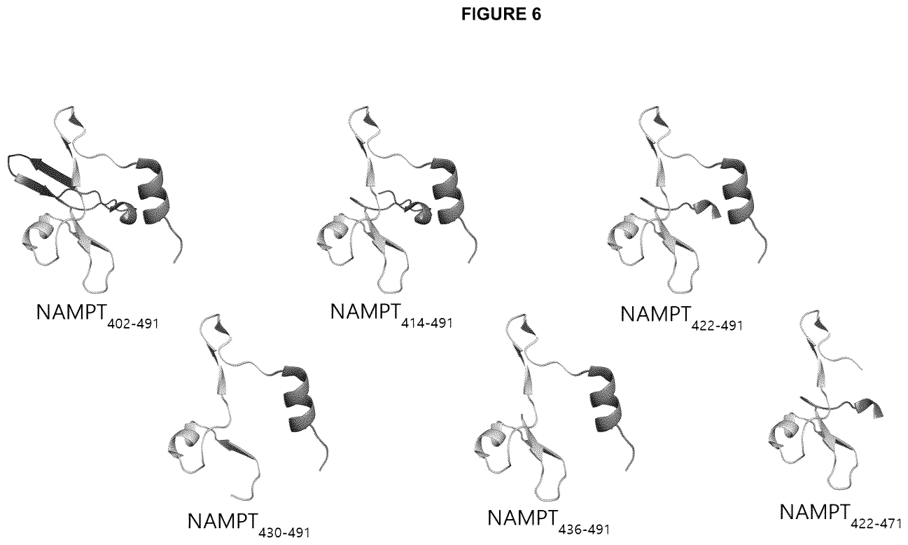

To further elucidate the active fragment of NAMPT cytokine finger, the inventors investigated additional truncations of NAMPT₄₀₂₋₄₉₁, NAMPT₄₁₄₋₄₉₁, NAMPT₄₂₂₋₄₉₁, NAMPT₄₃₀₋₄₉₁, NAMPT₄₃₆₋₄₉₁ and NAMPT₄₂₂₋₄₇₁ (NAMPT variants, also referred to herein as hNAMPTcif, hNAMPTcif-T1, hNAMPTcif-T2, hNAMPTcif-T3, hNAMPTcif-T4 and hNAMPTcif-T5 respectively). Amino acid numbering corresponds to full length NAMPT, e.g. SEQ ID NO: 19. The predicted structure of the NAMPT variants are shown in FIG. 6.

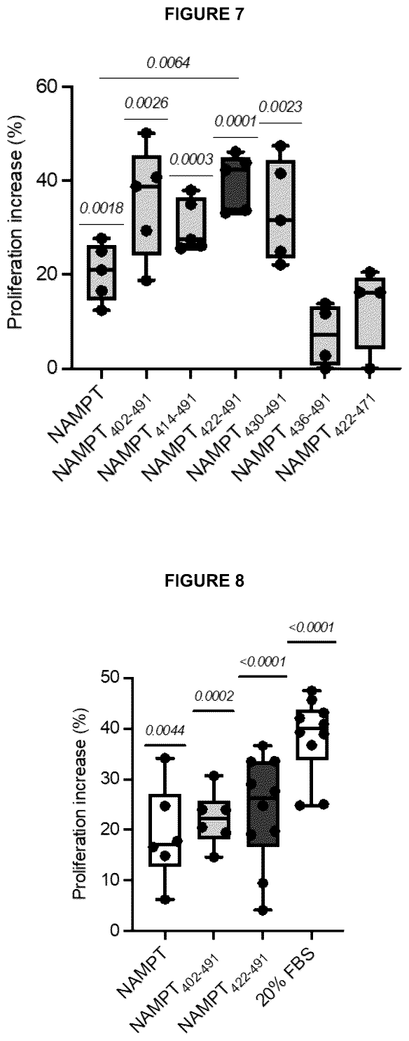

C2C12 mouse myoblasts were treated with 20 nM full length NAMPT (NAMPT) or NAMPT variants for 48 hours and muscle progenitor proliferation was assessed. NAMPT variants NAMPT₄₀₂₋₄₉₁, NAMPT₄₁₄₋₄₉₁ and NAMPT₄₃₀₋₄₉₁ all exhibited enhanced cellular proliferation compared to full-length NAMPT (FIG. 7). NAMPT₄₂₂₋₄₉₁ displayed the highest cellular proliferation compared to all NAMPT variants tested. In particular, there was a statistically significant increase in proliferation in muscle progenitor cells treated with NAMPT₄₂₂₋₄₉₁ compared to full-length NAMPT. Interestingly, NAMPT₄₃₆₋₄₉₁ and NAMPT₄₂₂₋₄₇₁ exhibited lower cellular proliferation of muscle progenitor cells compared to full-length NAMPT and the other NAMPT variants.

### Example 9—Human NAMPT Variants Stimulate Human Satellite Cell Proliferation

The inventors next sought to compare the activity of full length NAMPT, NAMPT₄₀₂₋₄₉₁ and NAMPT₄₂₂₋₄₉₁ on human satellite cell proliferation. Human primary satellite cells were treated with 20 nM full length NAMPT (NAMPT) or NAMPT₄₀₂₋₄₉₁ and NAMPT₄₂₂₋₄₉₁ for 48 hours.

NAMPT₄₀₂₋₄₉₁ exhibited increased satellite cell proliferation compared to full length NAMPT, with NAMPT₄₂₂₋₄₉₁ demonstrating even greater satellite cell proliferation compared to both full length NAMPT and NAMPT₄₀₂₋₄₉₁ (FIG. 8).

### Example 10—Human NAMPT Variants Stimulate Human Endothelial Cell Proliferation

Similarly, the inventors next investigated the activity of full length NAMPT, NAMPT₄₀₂₋₄₉₁ and NAMPT₄₂₂₋₄₉₁ on human endothelial cell proliferation. Human endothelial cells were derived from umbilical vein and treated with 20 nM full length NAMPT (NAMPT) or NAMPT variants for 48 hours.

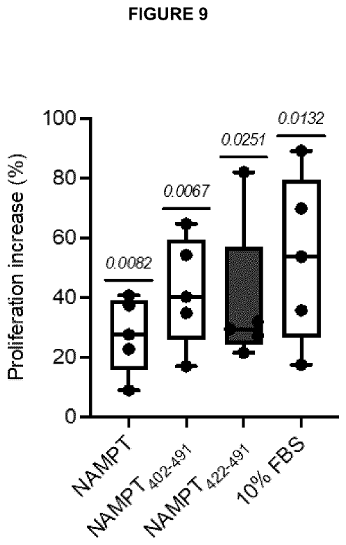

NAMPT₄₀₂₋₄₉₁ displayed increased endothelial cell proliferation compared to full length NAMPT and NAMPT₄₂₂₋₄₉₁. Full length NAMPT and NAMPT₄₂₂₋₄₉₁, however, still stimulated endothelial cell proliferation above negative control (FIG. 9).

### Example 10—Minimal Versions of NAMPT Protein Enhance Proliferation in Response to Muscle Injury in Zebrafish Larvae

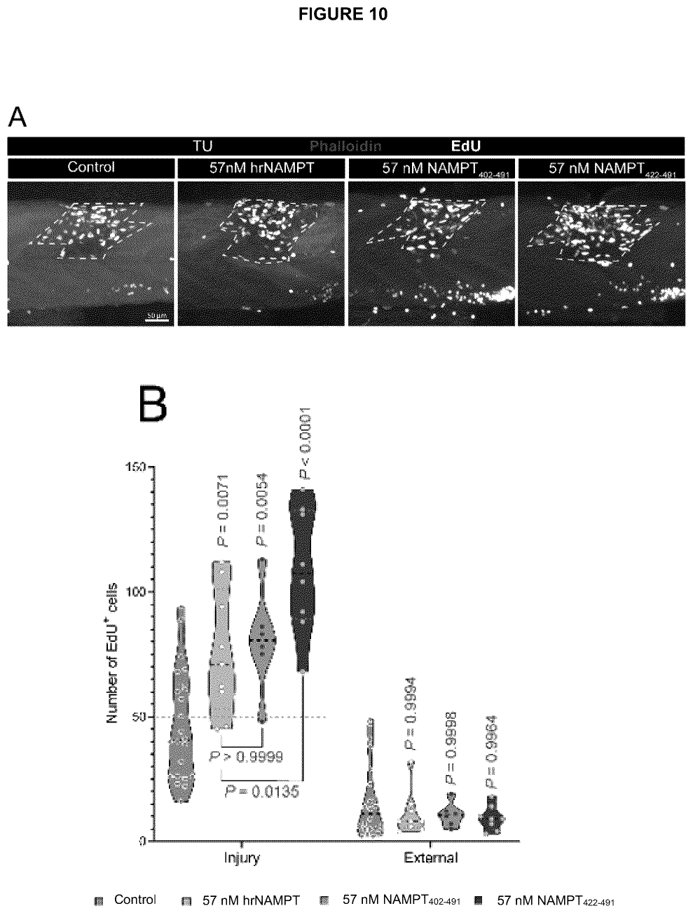

Next, the inventors tested the ability of NAMPT variants to stimulate cell proliferation in an in vivo zebrafish muscle injury response model. Treatment with NAMPT₄₀₂₋₄₉₁ and NAMPT₄₂₂₋₄₉₁ following needle-stick muscle injury to the zebrafish lavae induced a significant increase in cell proliferation within the injury zone (FIG. 10). The smallest version of NAMPT (NAMPT₄₂₂₋₄₉₁), stimulated cell proliferation specifically in the wound at significantly higher levels as compared to human recombinant-NAMPT (hrNAMPT).

Collectively, these findings indicate that exogenously supplied NAMPT protein stimulates muscle repair in the context of an acute injury of zebrafish larvae in a similar manner to the results we describe above for adult mammalian muscle. It also reinforces the finding that it is the secreted form of NAMPT that is active in both these in vivo settings.

1. A polypeptide comprising, consisting essentially of or consisting of a C-terminal portion of NAMPT comprising a truncated cytokine finger motif (cif).

2. The polypeptide according to claim 1, wherein the only amino acid sequence of the polypeptide that is derived from or has homology or identity to the NAMPT protein is the truncated cif motif.

3. The polypeptide according to claim 1 or 2, wherein the polypeptide binds to CCR5 and/or stimulates muscle progenitor proliferation.

4. The polypeptide according to any one of claims 1 to 3, wherein the only amino acid sequence of the polypeptide that binds to CCR5 and/or stimulates muscle progenitor stimulation is the truncated cif motif.

5. The polypeptide according to any one of claims 1 to 4, wherein the truncation of the cif motif is an N-terminal truncation.

6. The polypeptide according to any one of claims 1 to 4, wherein the truncation of the cif motif is a C-terminal truncation.

7. The polypeptide according to any one of claims 1 to 6, wherein the N- and/or C-terminal truncation is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids.

8. The polypeptide according to any one or claims 1 to 7, wherein the truncation is of the N-terminal residues 1-12 of the amino acid sequence of, or equivalent to, SEQ ID NO: 1.

9. The polypeptide according to any one or claims 1 to 7, wherein the truncation is of the N-terminal residues 1-20 of the amino acid sequence of, or equivalent to, SEQ ID NO: 1.

10. The polypeptide according to any one or claims 1 to 7, wherein the truncation is of the N-terminal residues 1-28 of the amino acid sequence of, or equivalent to, SEQ ID NO: 1.

11. The polypeptide according to any one or claims 1 to 7, wherein the truncation is of the N-terminal residues 1-35 of the amino acid sequence of, or equivalent to, SEQ ID NO: 1.

12. The polypeptide according to any one or claims 1 to 11, wherein the cif motif comprises or consists of the amino acid sequence set out in any one of SEQ ID Nos: 1, 2 or 3.

13. The polypeptide according to any one or claims 1 to 12, wherein the cif motif comprises or consists of the amino acid sequence set out in SEQ ID No: 1.

14. The polypeptide according to any one or claims 1 to 13, wherein the polypeptide comprises, consists essentially of or consists of an amino acid sequence that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 4, 5, 6, 7, 8, 9, 10 or 11.

15. The polypeptide according to any one or claims 1 to 13, wherein the polypeptide comprises, consists essentially of or consists of an amino acid sequence that is equal to, or at least, 70%, 75%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID Nos: 4, 5, 6, 7, 8, 9, 10 or 11.

16. The polypeptide according to any one or claims 1 to 15, wherein the polypeptide is equal to, or less than, about 110, about 109, about 108, about 107, about 106, about 105, about 104, about 103, about 102, about 101, about 100, about 99, about 98, about 97, about 96, about 95, about 94, about 93, about 92, about 91, about 90, about 89, about 88, about 87, about 86, about 85, about 84, about 83, about 82, about 81, about 80, about 79, about 78, about 77, about 76, about 75, about 74, about 73, about 72, about 71, about 70, about 69, about 68, about 67, about 66, about 65, about 64, about 63, about 62, about 61, about 60, about 59, about 58, about 57, or about 56 amino acids in length.

17. The polypeptide according to any one or claims 1 to 15, wherein the polypeptide is equal to, or less than, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, or 56 amino acids in length.

18. The polypeptide according to any one or claims 1 to 17, wherein the polypeptide comprises an amino acid sequence of any one of SEQ ID Nos: 4, 5, 6, 7, 8, 9, 10 or 11 or comprises an amino acid sequence having 1, 2, 3, 4, 5, 6, 7 or 8 conservative or non-conservative amino acid substitutions, deletions or additions to the above sequences, and retains CCR5 or tissue stem cell interacting activity.

19. The polypeptide according to any one or claims 1 to 17, wherein the polypeptide consists of an amino acid sequence of any one of SEQ ID Nos: 4, 5, 6, 7, 8, 9, 10 or 11 or consists of an amino acid sequence having 1, 2, 3, 4, 5, 6, 7 or 8 conservative or non-conservative amino acid substitutions, deletions or additions to the above sequences, and retains CCR5 or tissue stem cell interacting activity.

20. The polypeptide according to any one or claims 1 to 17, wherein the polypeptide comprises an amino acid sequence of any one of SEQ ID Nos: 4, 5, 6, 7, 8, 9, 10 or 11.

21. The polypeptide according to any one or claims 1 to 17, wherein the polypeptide consists essentially of an amino acid sequence of any one of SEQ ID Nos: 4, 5, 6, 7, 8, 9, 10 or 11.

22. The polypeptide according to any one or claims 1 to 17, wherein the polypeptide consists of an amino acid sequence of any one of SEQ ID Nos: 4, 5, 6, 7, 8, 9, 10 or 11.

23. The polypeptide according to any one or claims 1 to 17, wherein the polypeptide consists of an amino acid sequence of SEQ ID NO: 4.

24. The polypeptide according to any one or claims 1 to 17, wherein the polypeptide consists of an amino acid sequence of SEQ ID NO: 6.

25. The polypeptide according to any one or claims 1 to 17, wherein the polypeptide consists of an amino acid sequence of SEQ ID NO: 8.

26. The polypeptide according to any one or claims 1 to 17, wherein the polypeptide consists of an amino acid sequence of SEQ ID NO: 10.

27. The polypeptide according to any one of claims 1 to 26, wherein the polypeptide is monomeric form.

28. The polypeptide according to any one of claims 1 to 26, wherein the polypeptide is dimeric form.

29. The polypeptide according to any one of claims 1 to 26, wherein the polypeptide is homodimeric form.

30. A fusion protein comprising, consisting essentially of or consisting of a polypeptide according to any one of claims 1 to 29 and a tissue delivery or retention enhancing moiety.

31. The fusion protein according to claim 30, wherein the tissue delivery or retention enhancing moiety is an extracellular matrix (ECM) binding moiety.

32. The fusion protein according to claim 30 or 31, wherein the ECM binding moiety binds to any one or more of the following ECM molecules: collagen, fibronectin, tenascin C, osteopontin, fibrinogen, and heparan sulfate.

33. The fusion protein according to any one of claims 30 to 32, wherein the ECM binding moiety is derived from placenta growth factor (PIGF), amphiregulin (Areg), collagenase or von Willebrand factor (vWF).

34. The fusion protein according to any one of claims 30 to 33, wherein the ECM binding moiety comprises, consists essentially of or consists of positively charged amino acid residues.

35. The fusion protein according to claim 34, wherein the positively charged residues comprises, consists essentially of or consists of RRRPK, RKKK, KRRR or SEQ ID NO: 12 and 13.

36. The fusion protein according to any one of claims 30 to 35, wherein the ECM binding moiety comprises, consists essentially of or consist of an amino acid sequence that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 12 to 16, wherein the ECM binding moiety binds to one or more ECM proteins with the same affinity, an affinity not significantly different, or an affinity of at least 80%, 95%, 90%, 91%, 92%, 93%, 94%, 95%, 96%, 97%, 98% or 99%, an ECM binding moiety of anyone or SEQ ID Nos: 12 to 16 from which it was derived.

37. The fusion protein according to any one of claims 30 to 36, wherein the ECM binding moiety comprises, consists essentially of or consist of an amino acid sequence of any one of SEQ ID NOs: 12 to 16.

38. The fusion protein according to any one of claims 30 to 37, wherein the fusion protein comprises, consists essentially of or consists of the amino acid sequence of any one of SEQ ID NO: 12 to 16 fused, linked, or linked directly to the amino acid sequence of any one of SEQ ID Nos: 4 to 11.

39. A fusion protein comprising, consisting essentially of or consisting of an ECM binding moiety and a full length cytokine finger motif of NAMPT, wherein the ECM binding moiety binds to any one or more of the following ECM molecules: collagen, fibronectin, tenascin C, osteopontin, fibrinogen, and heparan sulfate.

40. The fusion protein according to claim 39, wherein the ECM binding moiety is derived from placenta growth factor (PIGF), amphiregulin (Areg), collagenase or von Willebrand factor (vWF).

41. The fusion protein according to claim 39 or 40, wherein the ECM binding moiety comprises, consists essentially of or consists of positively charged amino acid residues.

42. The fusion protein according to claim 41, wherein the positively charged residues comprises, consists essentially of or consists of RRRPK, RKKK, KRRR or SEQ ID NO: 12 and 13.

43. The fusion protein according to any one of claims 39 to 42, wherein the ECM binding moiety comprises, consists essentially of or consist of an amino acid sequence that is equal to, or at least, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identical to an amino acid sequence of any one of SEQ ID Nos: 12 to 16, wherein the ECM binding moiety binds to one or more ECM proteins with the same affinity, an affinity not significantly different, or an affinity of at least 80%, 95%, 90%, 91%, 92%, 93%, 94%, 95%, 96%, 97%, 98% or 99%, an ECM binding moiety of anyone or SEQ ID Nos: 12 to 16 from which it was derived.

44. The fusion protein according to any one of claims 39 to 43, wherein the ECM binding moiety comprises, consists essentially of or consist of an amino acid sequence of any one of SEQ ID NOs: 12 to 16.

45. The fusion protein according to any one of claims 39 to 43, wherein the cytokine finger motif of NAMPT comprises, consists essentially of or consists of the amino acid sequence of SEQ ID NO: 1.

46. A nucleic acid encoding the polypeptide according to any one of claims 1 to 29.

47. A nucleic acid encoding the fusion polypeptide according to any one of claims 30 to 45.

48. A vector comprising the nucleic acid according to claim 46 or 47.

49. A cell comprising a nucleic acid according to claim 46 or 47, or a vector according to claim 48.

50. A composition comprising a polypeptide, fusion protein, nucleic acid, vector, or cell according to any one of the preceding claims and a pharmaceutically acceptable carrier, diluent or excipient.

51. A composition according to claim 50 further comprising one, two or all of (a) a tissue stem cell (such as a satellite cell) or precursor therefore or progeny thereof, (b) a macrophage or a precursor therefore or progeny thereof, and (c) a scaffold or retentive material.

52. A method of stimulating proliferation of a stem cell, such as satellite cell proliferation, the method comprising administering to a cell or subject an effective amount of the polypeptide, fusion protein, nucleic acid, vector, cell or composition according to any one of claims 1 to 51, thereby stimulating proliferation of a stem cell.

53. A method of stimulating muscle tissue regeneration in a subject, the method comprising administering to a muscle of the subject an effective amount of the polypeptide, fusion protein, nucleic acid, vector, cell or composition according to any one of the preceding claims, thereby stimulating muscle tissue regeneration.

54. A method of stimulating muscle tissue regeneration, the method comprising administering to a muscle an effective amount of a composition comprising a cell comprising or encoding the polypeptide or fusion protein according to any one of claims 1 to 45, and optionally a component that enhances delivery to or retention in the muscle, wherein the polypeptide or fusion protein binds to satellite cells and stimulates myoblast proliferation and muscle regeneration.

55. The method of claim 54, wherein the cell is a macrophage.

56. The method of claim 55, wherein the macrophage is isolated from tissue.

57. The method of claim 56, wherein the macrophage is induced from stem cells such as bone marrow precursors or iPSC.

58. A polypeptide, fusion protein, nucleic acid, vector, cell or composition according to any one of claims 1 to 51 for use as a medicament or for use in therapy.

59. A polypeptide, fusion protein, nucleic acid, vector, cell or composition according to any one of claims 1 to 51 for use in stimulating muscle stem cell proliferation.

60. Use of a polypeptide, fusion protein, nucleic acid, vector, cell or composition according to any one of claims 1 to 51 in the manufacture of a medicament for stimulating muscle regeneration or in stem cell therapy.

61. A method of stimulating muscle tissue regeneration in a subject where inflammation is undesirable, the method comprising administering to a muscle an effective amount of a polypeptide, fusion protein, nucleic acid, vector, composition or cell of any one of claims 1 to 51, thereby stimulating muscle tissue regeneration in this subject.

62. The method according to claim 61, wherein the inflammation that is undesirable is inflammation mediated by TLR activation.

63. The method according to claim 62, wherein the TLR activation is TLR4 activation.

64. A method of treating an inflammatory myopathy in a subject, the method comprising administering to a muscle of the subject an effective amount of a polypeptide, fusion protein, nucleic acid, vector, cell or composition according to any one of claims 1 to 51, thereby treating an inflammatory myopathy.

65. The method according to claim 64, wherein the inflammatory myopathy is polymyositis, dermatomyositis, inclusion body myositis or necrotizing autoimmune myopathy.


SEQUENCE LISTING (35 sequences)

SEQ ID NO: 1 - protein, 90 aa, Homo sapiens
SYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 2 - protein, 91 aa, synthetic construct
CSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 3 - protein, 90 aa, Mus musculus
SYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 4 - protein, 78 aa, synthetic construct
FKDPVADPNKRSKKGRLSLHRTPAGNFVTLEEGKGDLEEYGQDLLHTVFKNGKVTKSYSFDEIRKNAQLNIELEAAHH

SEQ ID NO: 5 - protein, 93 aa, synthetic construct
HHHHHHHHENLYFQGFKDPVADPNKRSKKGRLSLHRTPAGNFVTLEEGKGDLEEYGQDLLHTVFKNGKVTKSYSFDEIRKNAQLNIELEAAHH

SEQ ID NO: 6 - protein, 70 aa, synthetic construct
NKRSKKGRLSLHRTPAGNFVTLEEGKGDLEEYGQDLLHTVFKNGKVTKSYSFDEIRKNAQLNIELEAAHH

SEQ ID NO: 7 - protein, 85 aa, synthetic construct
HHHHHHHHENLYFQGNKRSKKGRLSLHRTPAGNFVTLEEGKGDLEEYGQDLLHTVFKNGKVTKSYSFDEIRKNAQLNIELEAAHH

SEQ ID NO: 8 - protein, 62 aa, synthetic construct
LSLHRTPAGNFVTLEEGKGDLEEYGQDLLHTVFKNGKVTKSYSFDEIRKNAQLNIELEAAHH

SEQ ID NO: 9 - protein, 77 aa, synthetic construct
HHHHHHHHENLYFQGLSLHRTPAGNFVTLEEGKGDLEEYGQDLLHTVFKNGKVTKSYSFDEIRKNAQLNIELEAAHH

SEQ ID NO: 10 - protein, 56 aa, synthetic construct
PAGNFVTLEEGKGDLEEYGQDLLHTVFKNGKVTKSYSFDEIRKNAQLNIELEAAHH

SEQ ID NO: 11 - protein, 71 aa, synthetic construct
HHHHHHHHENLYFQGPAGNFVTLEEGKGDLEEYGQDLLHTVFKNGKVTKSYSFDEIRKNAQLNIELEAAHH

SEQ ID NO: 12 - protein, 19 aa, synthetic construct
RRRPKGRGKRRREKQRPTD

SEQ ID NO: 13 - protein, 13 aa, synthetic construct
RKKKGGKNGKNRR

SEQ ID NO: 14 - protein, 10 aa, synthetic construct
WREPSFMALS

SEQ ID NO: 15 - protein, 196 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 16 - protein, 7 aa, synthetic construct
TKKTLRT

SEQ ID NO: 17 - protein, 110 aa, synthetic construct
RRRPKGRGKRRREKQRPTDCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 18 - protein, 104 aa, synthetic construct
RKKKGGKNGKNRRCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 19 - protein, 491 aa, Homo sapiens [sequence omitted]

SEQ ID NO: 20 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 21 - DNA, 272 bp, synthetic construct
  source 1..272: mol_type = other DNA
agctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcatta

SEQ ID NO: 22 - DNA, 271 bp, synthetic construct
  source 1..271: mol_type = other DNA
agctatgttgtaaccaatggccttggggttaatgtgtttaaggacccagttgctgatcccaacaaaaggtcaaaaaagggccggttatctttacataggacaccagcggggaactttgttacacttgaagaaggaaaaggagaccttgaggaatatggccatgatcttctccatacggttttcaagaatgggaaggtgacaaaaagctactcatttgatgaagtcagaaaaaatgcacagctgaacatcgagcaggacgtggcacctcatt

SEQ ID NO: 23 - DNA, 1475 bp, synthetic construct
  source 1..1475: mol_type = other DNA
atgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcatta

SEQ ID NO: 24 - DNA, 1474 bp, synthetic construct
  source 1..1474: mol_type = other DNA
atgaatgctgcggcagaagccgagttcaacatcctgctggccaccgactcgtacaaggttactcactataaacaatacccacccaacacaagcaaagtttattcctactttgaatgccgtgaaaagaagacagaaaactccaaagtaaggaaggtgaaatacgaggaaacagtattttatgggttgcagtacattcttaataagtacttaaaaggtaaagtagtgaccaaagagaaaatccaggaggccaaagaagtgtacagagaacatttccaagatgatgtctttaacgaaagaggatggaactacatccttgagaaatacgatggtcatctcccgattgaagtaaaggctgttcccgagggctctgtcatccccagagggaacgtgctgttcacagtggaaaacacagacccagagtgctactggcttaccaattggattgagactattcttgttcagtcctggtatccaattacagtggccacaaattccagagaacagaagagaatactggccaaatatttgttagaaacctctggtaacttagatggtctggaatacaagttacatgactctggttacagaggagtctcttcgcaagagactgctggcataggggcatctgctcatttggttaacttaaaaggaacagatactgtggcgggaattgctctaattaaaaaatactatgggacaaaagatcctgttccaggctattctgttccagcagcagagcacagtaccataacggcttgggggaaagaccatgagaaagatgcttttgaacacatagtaacacagttctcatcagtgcctgtgtctgtggtcagcgatagctatgacatttataatgcgtgtgagaaaatatggggtgaagacctgagacatctgatagtatcgagaagtacagaggcaccactaatcatcagacctgactctggaaatcctcttgacactgtattgaaggtcttagatattttaggcaagaagtttcctgttactgagaactcaaaaggctacaagttgctgccaccttatcttagagtcattcaaggagatggcgtggatatcaatactttacaagagattgtagagggaatgaaacaaaagaagtggagtatcgagaatgtctccttcggttctggtggcgctttgctacagaagttaacccgagacctcttgaattgctccttcaagtgcagctatgttgtaaccaatggccttggggttaatgtgtttaaggacccagttgctgatcccaacaaaaggtcaaaaaagggccggttatctttacataggacaccagcggggaactttgttacacttgaagaaggaaaaggagaccttgaggaatatggccatgatcttctccatacggttttcaagaatgggaaggtgacaaaaagctactcatttgatgaagtcagaaaaaatgcacagctgaacatcgagcaggacgtggcacctcatt

SEQ ID NO: 25 - DNA, 237 bp, synthetic construct
  source 1..237: mol_type = other DNA
ttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 26 - DNA, 213 bp, synthetic construct
  source 1..213: mol_type = other DNA
aacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 27 - DNA, 189 bp, synthetic construct
  source 1..189: mol_type = other DNA
ctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 28 - DNA, 171 bp, synthetic construct
  source 1..171: mol_type = other DNA
ccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 29 - DNA, 303 bp, synthetic construct
  source 1..303: mol_type = other DNA
tggcgcgaaccgagctttatggcgctgagctcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 30 - DNA, 861 bp, synthetic construct
  source 1..861: mol_type = other DNA
tgcagccagccgctggatgtgattctgctgctggatggcagcagcagctttccggcgagctattttgatgaaatgaaaagctttgcgaaagcgtttattagcaaagcgaacattggcccgcgcctgacccaggtgagcgtgctgcagtatggcagcattaccaccattgatgtgccgtggaacgtggtgccggaaaaagcgcatctgctgagcctggtggatgtgatgcagcgcgaaggcggcccgagccagattggcgatgcgctgggctttgcggtgcgctatctgaccagcgaaatgcatggcgcgcgcccgggcgcgagcaaagcggtggtgattctggtgaccgatgtgagcgtggatagcgtggatgcggcggcggatgcggcgcgcagcaaccgcgtgaccgtgtttccgattggcattggcgatcgctatgatgcggcgcagctgcgcattctggcgggcccggcgggcgatagcaacgtggtgaaactgcagcgcattgaagatctgccgaccatggtgaccctgggcaacagctttctgcataaactgtgcagcggctttgtgcgcatttgcaccggctcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 31 - DNA, 294 bp, synthetic construct
  source 1..294: mol_type = other DNA
accaaaaaaaccctgcgcacctcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 32 - DNA, 273 bp, synthetic construct
  source 1..273: mol_type = other DNA
tcttacgttgttaccaacggtctgggtatcaacgttttcaaagacccggttgctgacccgaacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttactctttcgacgaaatccgtaaaaacgctcagctgaacatcgaactggaagctgctcaccactaa

SEQ ID NO: 33 - protein, 49 aa, synthetic construct
NKRSKKGRLSLHRTPAGNFVTLEEGKGDLEEYGQDLLHTVFKNGKVTKS

SEQ ID NO: 34 - protein, 64 aa, synthetic construct
HHHHHHHHENLYFQGNKRSKKGRLSLHRTPAGNFVTLEEGKGDLEEYGQDLLHTVFKNGKVTKS

SEQ ID NO: 35 - DNA, 150 bp, synthetic construct
  source 1..150: mol_type = other DNA
aacaaacgttctaaaaaaggtcgtctgtctctgcaccgtaccccggctggtaacttcgttaccctggaagaaggtaaaggtgacctggaagaatacggtcaggacctgctgcacaccgttttcaaaaacggtaaagttaccaaatcttac